(19) United States
(12) Patent Application Publication
(10) Pub. No.: US 2025/0345493 A1
(43) Pub. Date: Nov. 13, 2025

(54) ANTIMICROBIAL COMPOSITIONS: METHODS OF MAKING AND USING THE SAME

(71) Applicant: ZONOVA TECHNOLOGIES LTD, Kent (GB)

(72) Inventors: Frances HEALE, Kent (GB); Georgia FLEET, Kent (GB)

(73) Assignee: ZONOVA TECHNOLOGIES LTD, Kent (GB)

(21) Appl. No.: 19/201,759

(22) Filed: May 7, 2025

Related U.S. Application Data
(60) Provisional application No. 63/643,772, filed on May 7, 2024.

(30) Foreign Application Priority Data
Oct. 21, 2024 (GB) 2415460.1

(51) Int. Cl. A61L 29/16; A61L 29/02

(52) U.S. Cl. CPC A61L 29/16; A61L 29/02; A61L 2300/102; A61L 2300/406; A61L 2400/12

(57) ABSTRACT

The invention provides antimicrobial compositions comprising a metallic nanoparticle and a photosensitizer mixed to form a metallic nanoparticle-photosensitizer mixture which has activity in dark conditions without needing a light source. The invention also provides methods of preparing an antimicrobial metallic nanoparticle-photosensitizer mixture compositions and methods of encapsulating a polymer with the antimicrobial metallic nanoparticle-photosensitizer mixture compositions disclosed by the invention.

### CROSS REFERENCE TO RELATED APPLICATIONS

This application claims the benefit of and priority to U.S. Provisional Application No. 63/643,772 filed on May 7, 2024, and Great Britain Patent Application No. 2415460.1, filed on Oct. 21, 2024, which are incorporated by reference in their entirety.

### FIELD

This invention relates generally to antimicrobial compositions and uses therefore, wherein the antimicrobial compositions are used in objects susceptible to risks of infection that can be harmful to humans or animals, such as in a medical environment.

### BACKGROUND

Patients who receive implantable devices, including total joint prosthetics, vascular devices, pacemakers, fracture fixation devices, or who undergo surgery in general, are at risk of being contaminated with bacteria, including those in the biofilm phenotype. Antimicrobial therapies that are currently in clinical use remain limited in their ability to effectively treat and prevent biofilm-related infections, in particular those that accompany the use of implanted devices.

Photosensitizers, such as toluidine blue O, act as light-activated antimicrobial agents. Although they may have no antimicrobial activity at low concentrations in the dark, when irradiated with light of a certain wavelength (such as 633 nm for toluidine blue O) they can kill a wide range of microbes. The killing of microbes is thought to be due to the singlet oxygen produced on irradiation of the compound. There is considerable interest in enhancing the activity of existing photosensitizers.

U.S. Pat. No. 8,580,309B2 describes antimicrobial mixtures comprising charge-stabilized metallic nanoparticles and a photosensitizer, and their use as light activated antimicrobials. The authors described metallic nanoparticle-ligand-photosensitizer conjugates for use as light activated antimicrobials capable of killing or preventing the growth of microbes on surfaces in a medical environment. This antimicrobial technology relies on nanoparticles to enhance the natural antimicrobial activity of the photosensitizer dye, which haven't in the past exhibited their own antimicrobial activity. However, these photosensitizer dyes are activated by light, and therefore, are not active in the dark. Medical devices implanted in living subjects often sit within the body in the dark and cannot be activated by light. Therefore, there is a need for antimicrobial compositions that can be activated within a subject's body, without a light source.

Presented herein are antimicrobial compositions that are active in light conditions, as well as dark conditions without a need for a light source.

### SUMMARY

In accordance with the purpose(s) of this invention, as embodied and broadly described herein, this invention, in some aspects, relates to antimicrobial compositions comprising one or more metallic nanoparticles selected from zinc, silver, copper, gold nanoparticles, or alloys of two or more of these metals; and one or more photosensitizers selected from porphyrins, chlorins, dyes, or xanthenes; wherein the one or more metallic nanoparticles are metal oxide nanoparticles or alloys thereof, wherein the one or more metal oxide nanoparticles are exposed to a basic environment to form negatively charged metal ions or exposed to an acidic environment to form positively charged metal ions; and wherein the metallic nanoparticles and the photosensitizers are mixed to form a metallic nanoparticle-photosensitizer mixture having activity in light or dark conditions without needing a light source. In one embodiment, the metal oxide nanoparticles are zinc oxide, silver oxide, copper oxide, gold oxide nanoparticles, or alloys thereof.

In some embodiments, the metal oxide is a zinc oxide, silver oxide, copper oxide, gold oxide nanoparticles or alloys thereof, wherein the zinc oxide, silver oxide, copper oxide, gold oxide nanoparticles or alloys thereof are exposed to a basic environment to form negatively charged zinc oxide, silver oxide, copper oxide, or gold oxide ions that form complexes with positively charged ions, such as methylene blue and are very soluble in water, leading to much easier swell encapsulation in water-based solutions. In certain embodiments, the zinc oxide nanoparticle is exposed to a basic environment to form sodium zincate ions.

In another embodiment, the metal oxide nanoparticles are exposed to an acidic environment to form positively charged zinc, silver, copper, or gold ion, wherein the positively charged zinc, silver, copper, or gold ions are zinc chloride, silver chloride, copper chloride, or gold chloride.

In other embodiments, at least one photosensitizer is selected from a group comprising porphyrins (e.g. haematoporphyrin derivatives, deuteroporphyrin), phthalocyanines (e.g. zinc, silicon and aluminum phthalocyanines), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenothiaziniums (e.g. toluidine blue O, methylene blue, dimethylmethylene blue), phenazines (e.g. neutral red), acridines (e.g. acriflavine, proflavin, acridine orange, aminacrine), texaphyrins, cyanines (e.g. merocyanine 540), anthracyclins (e.g. adriamycin and epirubicin), pheophorbides, sapphyrins, fullerene, halogenated xanthenes (e.g. rose bengal), perylenequinonoid pigments (e.g. hypericin, hypocrellin), gilvocarcins, terthiophenes, benzophenanthridines, psoralens and riboflavin. Other possibilities are arianor steel blue, tryptan blue, crystal violet, azure blue cert, azure B chloride, azure 2, azure A chloride, azure B tetrafluoroborate, thionin, azure A eosinate, azure B eosinate, azure mix sicc. and azure II eosinate. In certain embodiments,

In some embodiments, the photosensitizer comprises methylene blue and/or rose bengal. In some particular embodiments, the metallic nanoparticle-photosensitizer mixture comprises a zinc-methylene blue, silver-methylene blue, copper-methylene blue, or gold-methylene blue conjugate. In other embodiments, the metallic nanoparticle-photosensitizer mixture comprises a zinc-rose bengal, silver-rose bengal, copper-rose bengal, or gold-rose bengal conjugate. In other embodiments, the negatively charged zinc oxide, silver oxide, copper oxide, or gold oxide ions are catalysts that activate the photosensitizer to react with a triplet oxygen (³O₂) to form a singlet oxygen (¹O₂) or free radicals, where the singlet oxygen or free radical exhibits antimicrobial effects.

In another aspect, the invention relates to a method for preparing an antimicrobial metallic nanoparticle-photosensitizer mixture described herein, comprising contacting a solution of charge-stabilized metallic nanoparticles with a solution of photosensitizer. In some embodiments, the metallic nanoparticle solution and the photosensitizer solution are aqueous solutions.

In yet another aspect, the invention relates to a method of encapsulating a polymer with the antimicrobial composition of claim 1 comprising: dissolving the metallic nanoparticle and photosensitizer dye in a solvent mixture; adding a pH agent is added to change the pH of a solution; placing the polymer into the solution wherein there is enough solution to cover the polymer; and incubating the polymer in the solution away from direct light; wherein polymer is incubated at room temperature in dark conditions for at least 8 to 48 hours. In other embodiments, the polymer is incubated at temperatures above room temperature.

In some embodiments, the solvent mixture comprises a water/acetone mixture in a ratio of 99:1, 90:10, 70:30, 60:40, or 50:50 of water to acetone. In other embodiments, the polymer comprises a long chain hydrophobic polymer selected from one or more of latex, polyamide, PVC, polyethylene, polyurethane, fluoropolymer, polycarbonate, polyesters, polyofins silicone, PTFE, PET, PMMA, HEMA, and combinations thereof. In some embodiments, at least one pH agent is a base selected from NaOH, sodium bicarbonate, potassium hydrogen carbonate, potassium hydroxide, lithium hydroxide, calcium hydroxide, strontium hydroxide, barium hydroxide, ammonia, ethylamine, pyridine, aniline, ethylamine, or ammonium hydroxide. In other embodiments, at least one pH agent is an acid selected from hydrochloric acid, phosphoric acid, nitric acid, perchloric acids, hydrobromic acid, sulfuric acid, hydroiodic acid, nitrous acid, formic acid, acetic acid, hydrocyanic acid, hydrogen and sulfide, hydrofluoric acid.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTIONS OF THE DRAWINGS

The accompanying drawings, which are incorporated in and constitute a part of this specification, illustrate (one) several embodiment(s) of the invention and together with the description, serve to explain the principles of the invention.

FIG. 1 is a bar graph showing the antimicrobial activity of a wound drain against methicillin-resistant resistant Staphylococcus aureus.

FIG. 2 is a bar graph showing the antimicrobial activity of a wound drain against multi-drug resistant Pseudomonas aeruginosa.

FIG. 3 shows a pH study in a 70/30 DIW/Acetone mix with ZnO/dye and polymer samples inside each, with incremental amounts of NaOH in each solution.

FIG. 4 shows swell encapsulation of rose Bengal (RB) in acid conditions using HCl as an acid source.

FIGS. 5A-5G show that multiple implantable medical devices including penrose drains—latex (FIG. 5A), suture imitation—polyamide (FIG. 5B), endotracheal tubes—PVC (FIG. 5C), urinary catheters—PVC (FIG. 5D), feeding tubes—PVC (FIG. 5E), endotracheal tubes—siliconized PVC (FIG. 5F) and winged infusion set—siliconized PVC (FIG. 5G) were successfully swell encapsulated with the methylene blue and ZnO antimicrobial mixtures.

FIG. 6 depicts the swell encapsulation of different polymers (polyurethane Tom Tiddle urinary catheter, right; 100% silicone Anifoley urinary catheter, middle; siliconized PVC Smiths Portex ET tube, left) with various photosensitisers, nanoparticles, photosensitiser concentrations, solvent ratios, pH adjusters, and pH adjuster concentrations, as denoted in Table 5.

FIGS. 7A and 7B depict the results of a solvent swell encapsulation experiment with polyolefin-based elastomer (POBE, Convatec, FIG. 7B) and latex (FIG. 7A) polymers, as described in Example 6, wherein latex swells easily in multiple solvents, whereas POBE swells only in 70/30 water/methyl ethyl ketone (MEK).

FIGS. 8A and 8B depict a polyurethane catheter (FIG. 8A) and a BD-Insyte polyurethane catheter (FIG. 8B).

FIGS. 9A-9EE depict results of swell encapsulation experiments described in Experiments 7 and 8, including POBE, MB+ZnO (FIG. 9A); POBE, MB+Cu (FIG. 9B); POBE, MB+Ag (FIG. 9C); POBE, MB only (FIG. 9D); POBE, ZnO only (FIG. 9E); latex, MB+Cu (FIG. 9F); latex, MB+Ag (FIG. 9G); latex, MB only (FIG. 9H); latex, ZnO only (FIG. 9I); PVC-T, MB+Cu (FIG. 9J); PVC-T, MB+Ag (FIG. 9K); PVC-T, MB only (FIG. 9L); PVC-T, ZnO only (FIG. 9M); PVC-T, RB+ZnO (FIG. 9N); PVC-T, MB+Cu, MEK as solvent (FIG. 9O); PVC-T, control (FIG. 9P); latex, RB+ZnO (FIG. 9Q); latex, MB+Cu, MEK as solvent (FIG. 9R); latex, control (FIG. 9S); PVC-C, unsterilized, MB+ZnO (FIG. 9T); PVC-C, Gamma sterilized, MB+ZnO (FIG. 9U); PVC-C, EtO sterilized, MB+ZnO (FIG. 9V); PVC-C, control (FIG. 9W); PVC-T, control (FIG. 9X); PVC-T, MB+ZnO (FIG. 9Y); PVC-T, MB+Cu (FIG. 9Z); PVC-T, RB+ZnO (FIG. 9AA); PVC-T, RB+Cu (FIG. 9BB); PVC-T, MB only (FIG. 9CC); PVC-T, ZnO only (FIG. 9DD); PVC-T, Cu only (FIG. 9EE), wherein PVC-C denotes PVC-based urinary catheter, PVC-T denotes PVC-based endotracheal tube, POBE indicates POBE-based urinary catheter, and latex denotes latex-based drain.

FIGS. 11A and 11B depict tensile testing of a non-sterilised PU material control as described in Experiment 10.

FIGS. 12A and 12B depict tensile testing of a sterilised PU material as described in Experiment 10.

FIGS. 13A and 13B depict tensile testing of a second sterilised PU material as described in Experiment 10.

### DETAILED DESCRIPTION

The present invention may be understood more readily by reference to the following detailed description of preferred embodiments of the invention and the Examples included therein and to the Figures and their previous and following description.

### I. Definitions

To facilitate an understanding of the principles and features of the various embodiments of the disclosure, various illustrative embodiments are explained herein. Although exemplary embodiments of the disclosure are explained in detail, it is to be understood that other embodiments are contemplated. Accordingly, it is not intended that the disclosure is limited in its scope to the details of construction and arrangement of components set forth in the description or examples. The disclosure is capable of other embodiments and of being practiced or carried out in various ways.

In describing the exemplary embodiments, specific terminology will be resorted to for the sake of clarity. As used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value.

As used herein, the term “dark conditions” means away from direct light sources. As used herein, the term “light source” refers to source of light such as sunlight, visible light, laser light or any other light of any specific wavelength. The term ‘dark conditions” may be used interchangeably with the term “in the dark.”

As used herein, the term “pH agent” refers to a chemical used to change the pH of a solution. A base may be used to make a solution basic. The basic pH agent may be selected from NaOH, sodium bicarbonate, potassium hydrogen carbonate, potassium hydroxide, lithium hydroxide, calcium hydroxide, strontium hydroxide, barium hydroxide, ammonia, ethylamine, pyridine, aniline, ethylamine, or ammonium hydroxide. An acid may be used to make a solution acidic. The acidic pH agent may be selected from hydrochloric acid, phosphoric acid, nitric acid, perchloric acids, hydrobromic acid, sulfuric acid, hydroiodic acid, nitrous acid, formic acid, acetic acid, hydrocyanic acid, hydrogen and sulfide, hydrofluoric acid.

As used herein, the term “photosensitizer” refers to a substance capable of absorbing light and transferring energy to desired reactants.

As used herein, the term “sterilisation” refers to a method for sterilizing medical devices and/or pharmaceuticals.

### II. Compositions

### A. Nanoparticle-Photosensitizer Conjugates

### 1. Metallic Nanoparticles

Provided herein are antimicrobial nanoparticle-photosensitizer compositions for swell encapsulation of implantable medical devices. Also provided is a process of making antimicrobial materials active in both light and dark conditions using swell encapsulation. The process involves the incorporation of a nanoparticle, in this case zinc oxide nanopowder (<50 nm) and a photosensitizer dye, methylene blue in this case, into existing polymeric materials by swell encapsulation.

Polymeric materials used herein may be selected from a group consisting of latex, polyamide, PVC, silicones, polyethylene, polyurethane, fluoropolymer, polycarbonate, polyesters, polyofins silicone, polypropylene, fluoropolymers like polytetrafluoroethylene (PTFE), perfluoroalkoxy alkanes (PFA), fluorinatedethylenepropylene (FEP), expanded polytetrafluoroethylene (ePTFE), polyethylene terephthalate (PET), Poly(methyl methacrylate) (PMMA), hydroxyethyl methacrylate (HEMA), and combinations thereof.

The term “nanoparticles” is generally understood to mean particles having a diameter of from about 1 to about 100 nm. In some embodiments, the nanoparticles used have a diameter of from about 1 to about 30 nm. In some embodiments, the nanoparticles have a diameter of from about 2 to about 5 nm. In other embodiments, the nanoparticles have a diameter of from about 10 to about 25 nm. In yet other embodiments, the nanoparticles have a diameter from about 15 to about 20 nm.

Nanoparticles typically, but not exclusively, comprise metals. They may also comprise alloys of two or more metals, or more complex structures such as core-shell particles, rods, stars, spheres or sheets. A core-shell particle may typically comprise a core of one substance, such as a metal, metal oxide or silica, surrounded by a shell of another substance, such as a metal, metal oxide or metal selenide. The term “metallic” as used herein is intended to encompass all such structures having a metallic outer surface.

In some embodiments, the outer surface of the metallic nanoparticles of the present invention comprises a main group metal or transition metal, such as cobalt. In other embodiments, the metallic nanoparticles comprise one or more of zinc, gold, silver and/or copper nanoparticles, and/or alloys of two or more of these metals. In some embodiments, the nanoparticles are zinc nanoparticles.

The metallic nanoparticles of the present invention may be chosen such that, when attached via the ligand to the photosensitizer to form the metallic nanoparticle-photosensitizer mixture, the mixture generates singlet oxygen and/or free radicals. In some embodiments, the mixture generates both singlet oxygen and free radicals.

Singlet oxygen generation may be measured by assay: several such methods are known to those skilled in the art, for example, photoluminescence. Free radical generation may be measured using electron proton resonance (EPR).

Examples of metallic nanoparticles that may be suitable are nanoparticles having a diameter greater than about 2 nm which exhibit plasmon resonance in the wavelength band of about 200 to about 1600 nm, i.e. covering the visible to near infrared bands. The plasmon resonance may be measured by UV spectroscopy. It may be seen for both the free and conjugated nanoparticle. In some embodiments, nanoparticles will exhibit plasmon resonance at wavelengths of from about 500 to about 600 nm.

Another property which may be used to help select a suitable nanoparticle is the molar extinction coefficient of the conjugated photosensitizer. When a photosensitizer is conjugated via a ligand to a suitable nanoparticle, the extinction coefficient of the photosensitizer may be enhanced, compared to the extinction coefficient that would be expected based on an equivalent concentration of the photosensitizer alone. Without wishing to be bound by theory, it is thought that this enhancement occurs because the photosensitizer coordinates to the surface of the nanoparticle. Thus, in order to select suitable nanoparticles, the extinction coefficient of the conjugate could be measured, using a spectrophotometer. Any enhancement is acceptable. In some embodiments, the extinction coefficient may range anywhere from about 2 to about 30 times or more; in others, it may range from about 5 to about 30 times or more; in others, it may range from about 10 to about 30 times or more, and in others, it may range from about 20 to about 30 times or more, compared to what is expected based on the same concentration of the unconjugated photosensitizer.

In some embodiments, the outer surface of the nanoparticles may comprise one or more of the group comprising zinc, gold, silver or copper. In some embodiments, the nanoparticles may comprise one or more of the group comprising zinc, or alloys thereof. Suitable alloys may also comprise one or more of other metals such as silver, copper, gold, aluminium, or combinations thereof.

In other embodiments, the nanoparticles described in the preceding paragraph comprise core-shell particles. It is possible for such core-shell particles to comprise a magnetic core or magnetic layer. An example of such a magnetic core-shell particle is a particle having a magnetic core and an outer shell which comprises gold. In some embodiments, the nanoparticles are zinc nanoparticles.

In some embodiments, the ligand of the metallic nanoparticle-ligand-photosensitizer conjugate is a water-solubilizing ligand. This means that the conjugate as a whole is water soluble at a concentration of at least about 1×10⁻⁸ M (mol dm⁻³) at room temperature (25° C.). In some embodiments, the conjugate is water soluble at a concentration of at least about 1×10⁻⁷ M. In other embodiments, the conjugate is water soluble at a concentration of at least about 1×10⁻⁶ M.

The concentration for determining water solubility may be measured by any appropriate method. Suitable methods include UV absorption, inductively coupled plasma mass spectrometry (ICP-MS), SQUID (superconducting quantum interference device) magnetometry, EPR or Raman spectroscopy.

In some embodiments, the ligands are water-solubilizing ligands selected from the group comprising one or more of: sulfur ligands, such as thiols (alkanethiols and aromatic thiols), xanthates, disulfides, dithiols, trithiols, thioethers, polythioethers, tetradentate thioethers, thioaldehydes, thioketones, thion acids, thion esters, thioamides, thioacyl halides, sulfoxides, sulfenic acids, sulfenyl halides, isothiocyanates, isothioureas or dithiocarbamates; selenium ligands, such as selenols (aliphatic or aromatic), selenides, diselenides, dialkyl-diselenides (for example octaneselenol-nanoparticle is obtained from dioctyl-diselenide), selenoxides, selenic acids or selenyl halides; tellurium ligands, such as tellurols (aliphatic or aromatic), tellurides or ditellurides; phosphorus ligands, such as phosphines or phosphine oxides; nitrogen ligands, such as alkanolamines or aminoacids; and other ligands such as carboxylate ligands (e.g. myristate), isocyanide, acetone and iodine.

In some embodiments, the water-solubilizing ligands are selected from the group comprising one or more of: 3-mercaptopropionic acid, 4-mercaptobutyric acid, 3-mercapto-1,2-propanediol, cysteine, methionine, thiomalate, 2-mercaptobenzoic acid, 3-mercaptobenzoic acid, 4-mercaptobenzoic acid, tiopronin, selenomethionine, 1-thio-beta-D-glucose, glutathione and ITCAE pentapeptide.

### 2. Antimicrobial Photosensitizers

A photosensitizer is a compound that can be excited by light of a specific wavelength. Thus, such a compound may have an absorption band in the ultraviolet, visible or infrared portion of the electromagnetic spectrum and, when the compound absorbs radiation within that band, it generates cytotoxic species, thereby exerting an antimicrobial effect. The effect may be due to the generation of singlet oxygen but the invention is not limited to photosensitizers that exhibit antimicrobial effects through generation of singlet oxygen.

It is a requirement of the present invention that the photosensitizer is chosen such that, when attached to the metallic nanoparticle-ligand core to form the conjugate, the conjugate generates singlet oxygen and/or free radicals. Preferably, the conjugated photosensitizer generates both singlet oxygen and free radicals without the presence of a light source. Singlet oxygen and free radical generation may be measured as described above.

Without wishing to be bound by theory, it is thought that the photosensitizer and nanoparticles are associated via dative covalent bonds, wherein the electrons are provided by, for example, S or N moieties on the photosensitizer.

In some embodiments, the photosensitizer demonstrates antimicrobial activity within a subjects body, without a need to be activated by a light source. In some embodiments, the photosensitizer is one or more photosensitizers selected from the group comprising: porphyrins (e.g. haematoporphyrin derivatives, deuteroporphyrin), phthalocyanines (e.g. zinc, silicon and aluminum phthalocyanines), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenothiaziniums (e.g. toluidine blue O, methylene blue, dimethylmethylene blue), phenazines (e.g. neutral red), acridines (e.g. acriflavine, proflavin, acridine orange, aminacrine), texaphyrins, cyanines (e.g. merocyanine 540), anthracyclins (e.g. adriamycin and epirubicin), pheophorbides, sapphyrins, fullerene, halogenated xanthenes (e.g. rose bengal), perylenequinonoid pigments (e.g. hypericin, hypocrellin), gilvocarcins, terthiophenes, benzophenanthridines, psoralens and riboflavin. Other possibilities are arianor steel blue, tryptan blue, crystal violet, azure blue cert, azure B chloride, azure 2, azure A chloride, azure B tetrafluoroborate, thionin, azure A eosinate, azure B eosinate, azure mix sicc. and azure II eosinate. In one embodiment, particularly preferred photosensitizers are methylene blue, or rose bengal

a. Organic Photosensitizers

The photosensitizers may be organic (including dyes, porphyrins, chlorins, furocoularins, xanthenes or monoterpene) or non-organic (including gaseous mercury, quantum dots, or nanowires/nanorods). In the disclosed invention, the photosensitizers are orgainic as they are favorable for absorption through the swelling process.

Amongst the list of organic photosensitizers, three main families with similar mechanisms can be identified.

i. Porphyrins and Chlorins.

In some embodiments, the photosensitizer is categorized as a porphyrin or chlorin photosensitizer. These photosensitizers include porphyrin, porfimer, temoprfin, and talaporfin sodium. Porphyrin and porphyrin-like molecules constitute the most common type of photosensitizer; they are all built around the porphyrin centre:

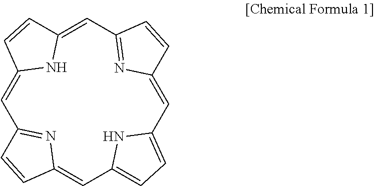 [SMILES: C1=Cc2cc3ccc(cc4nc(cc5ccc(cc1n2)[nH]5)C=C4)[nH]3]

This molecule, thanks to its highly conjugated structure can absorb photonic energy to enter an excited state commonly referred to as Sn (where n is the level of excitation, n=0 being the ground state). Each level of energy is stored in the higher molecular orbitals (moving from HOMO to LUMO) of the molecule and can be transferred to the orbital of triplet oxygen to excite it to a singlet state. Singlet oxygen is highly reactive to organic species and promotes the formation of peroxo bridges, which in term promote the formation of radicals, also know as ROS (Reactive oxygen species).

The absorption of photons by the porphyrin is determined by the energy bandgap between the HOMO and LUMO orbital. Various strategies can be developed to affect that bandgap, mostly by modifying the π-conjugated network and adding or replacing some heteroatoms. The resulting effect can determine the energy of the photon that is necessary to induce an excited state of porphyrin.

One of the strategies that can be used is the addition of a metal-based core in the centre of the porphyrin. This is commonly referred to as metalloporphyrin and is largely inspired by the structure of haems in haemoglobin. These molecules are generally synthesised ex situ but could be formed in situ under the right conditions.

It has been observed that under certain conditions, porphyrin could reach an excited state, not through the direct absorption of a photon but through the transfer of excitement via another molecules (enzymes, protein) (Afonso S G, Enríquez de Salamanca R, Batlle A M. Braz J Med Biol Res. 1999 March; 32 (3):255-66).

Therefore, it is possible that a cooperative effect between the porphyrin (or porphyrin-like compound) and a metal nanoparticle could, under the right circumstances, catalyse the formation of ROS without the need for photon absorption.

ii. Dyes and Xanthenes

In some embodiments, the photosensitizer is categorized as a dye and xanthenes photosensitizer. These photosensitizers include acridine orange, methyline blue, toludine blue O, Rose Bengal, and erythrosine. This family presents a structure conjugating τ bonds around a benzenic centre. In its oxidised state the dye's benzenic ring (one or more) is not saturated and instead displays a 1,4-cyclohexadiene structure linked to double bonds in the position 3,6. Those double bonds can be linked directly or via conjugation to a heteroatom presenting non-binding doublets (e.g. O, N, S). The driving mechanism for the reduction is the completion of the benzene ring and its stabilisation via aromaticity. This is initiated by the loss of 2 (or more) electrons and the bonding of one or more of the terminal heteroatoms to a H atom from the environment around the dye. This transformation ends with the more thermodynamically stable reduced form of the dye.

This process is entirely reversible should the reduced dye be exposed to a strong oxidiser (more thermodynamically reactive than the oxidised dye) and the dye being in a medium allowing proton exchange (e.g. water). This applies directly to the examples disclosed herein with methylene blue. Along with the aromatisation of a benzene ring the heteroatom can be involved in the closing (during the reduction) of a heterocycle. This applies directly to the examples disclosed herein with Rose Bengal (Hai, Yue & Feng, Shan & Wang, Lili & Ma, Yetao & Zhai, Yiran & Wu, Zijun & Zhang, Sichao & He, Xin. (2017). Coordination Mechanism and Bio-Evidence: Reactive γ-Ketoenal Intermediated Hepatotoxicity of Psoralen and Isopsoralen Based on Computer Approach and Bioassay. Molecules. 22. 1451. 10.3390/molecules22091451).

Because of this highly conjugated structure the band gap between HOMO and LUMO is such that the molecule can absorb a photon and enter an excited state. This energy can then be transferred to molecular oxygen to transform it from triplet oxygen to singlet oxygen, leading to the creation of ROS

However, it should also be mentioned that, because of their natural oxidative properties (and its reversible nature) this family of molecules can demonstrate antibacterial activity in the absence of a photon.

iii. Exotic Photosensitizers

In some embodiments, the photosensitizer is categorized as an exotic photosensitizer. These photosensitizers include azulene and psolaren. This family is less defined and regroups mostly seldomly used photosensitisers. These molecules tend to usually bind to certain sites under the effect of photon absorption and therefore are non-catalytic and don't present a specific mechanism. Based on their undefined properties, this category is no included in the disclosed compositions and methods. The photosensitizer categories that are used in the disclosed compositions and methods P are: Porphyrins, Chlorins, Dyes, and Xanthenes. Other photosensitisers can be listed and added individually based on their structure and mechanism of action.

The proportion of metallic nanoparticle: ligand: photosensitizer may vary. Typically, the nanoparticle comprises many atoms, only some of which have ligand molecules covalently bonded thereto. The number of photosensitizer molecules attached to each nanoparticle-ligand core may also vary. Typically, only some of the ligand molecules will have a photosensitizer molecule attached.

The metallic nanoparticle-photosensitizer mixture may also comprise further components. In some embodiments, it may have a targeting moiety associated with it. The targeting moiety can be associated with the conjugate via any suitable means, for example it may be attached to the nanoparticle core, to the ligand or to the photosensitizer. Such targeting moieties may be suitable, for example, for targeting specific microorganisms, or for targeting cancer cells. For example, they may be antibodies with specificity for the target organism or cancer cell. Other examples of targeting moieties include bacteriophages, protein A (targets Staphylococcus aureus) and bacterial cell-wall binding proteins or peptides.

The novel nanoparticle: photosensitizer compositions have been found to demonstrate particularly effective antimicrobial properties. Thus, all uses of mixtures as described herein apply to the novel compositions.

B. Metallic oxides and Methylene Blue Mixtures

Provided herein are antimicrobial compositions comprising zinc oxide and methylene blue mixtures.

### 1. Sodium Zincate

In basic conditions, Zinc oxide (ZnO) will react to form sodium zincate (Na₂Zn(OH)₄).

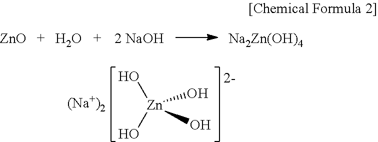

Unlike ZnO, zincate ions are very soluble in water, leading to much easier swell encapsulation in water-based solutions. Zincate ions can form hydrogen bonds, which could increase interaction with polymers. Zincate ions are also negative ions that can form complexes with positively charge ions such as methylene blue (MB).

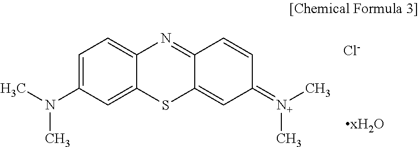

This could result in the formation of a [MB⁺]-[Zn(OH)₄²⁻]-[MB⁺] complex. Formation of these complexes are likely to increase polymer absorption (Yang, Q. et al. Carbohydrate Polymers 83(3): 1185-1191 (2011)).

### 2. Silver Oxide

The solubility of silver oxide has been studied by a large number of authors. Regardless of the form, Ag, or Ag₂O, the result is the same as Zn and ZnO. If the pH becomes more basic (>7) the oxides become more favourable (non-soluble species). If the pH becomes more acidic (<7) the Ag+ ion becomes more favourable (soluble species) (Molleman, B. Environmental science. Nano. 4(6):1314 (2017))

In another embodiment, other metallic oxides, such as silver oxide, copper oxide and gold oxides are exposed to basic conditions. In one embodiment, the silver oxide, copper oxide and gold oxides are exposed to NaOH to form ions that can form complexes with positively charge ions such as methylene blue and are very soluble in water, leading to much easier swell encapsulation in water-based solutions.

### 3. Metal Oxide With an Acid

Zinc oxide is an amphoteric oxide. It is nearly insoluble in water, but it will dissolve in most acids, such as hydrochloric acid:

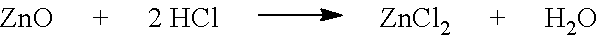

Solid zinc oxide will also dissolve in alkalis to give soluble zincates:

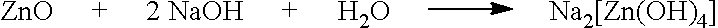

Therefore, the important step is choosing the correct conditions (i.e. basic or acidic) for the chosen photosensitizer. For rose bengal (negatively charged ion), acidic conditions are favorable. For methylene blue (positively charged ion), basic conditions are favorable.

The expected complex formations in the cases of methylene blue and rose bengal are
1. the formation of a [MB+]-[Zn(OH₄2−]-[MB+] complex.
2. the formation of a [RB−]-[Zn2+]-[RB−] complex.

A similar effect as with ZnO particles should be observed with Ag nanoparticles, in the case of Rose Bengal [RB]. Being mostly negatively charged in pH>3, this means complex [Ag⁺][RB⁻] are likely to form in pH<7. This can be extended to most metals, as they usually form positively charged ions when reacted with acid. (example: Mn, Cu, Fe) (Ford, Robert & Wilkin, Richard & Puls, Robert. (2007). Monitored Natural Attenuation of Inorganic Contaminants in Ground Water Volume 2—Assessment for Non-Radionuclides Including Arsenic, Cadmium, Chromium, Copper, Lead, Nickel, Nitrate, Perchlorate, and Selenium; Bruins, Jantinus. (2016). Manganese Removal from Groundwater. Role of Biological and Physico-Chemical Autocatalytic Processes; Werner Stumm, James J. Morgan, Aquatic Chemistry: Chemical Equilibria and Rates in Natural Waters, 3rd Edition, ISBN: 978-0-471-51185-4, October 1995).

In the case of Methylene blue far fewer species, metallic or oxides, form negatively charged hydroxides when in basic conditions. For instance, Ag does not form such species and therefore is unlikely to form a complex with MB in these conditions. However, for all metals which form negatively charged species when in basic condition, complexes such as the one observed with ZnO ([MB+]-[Zn(OH)42-]-[MB+]) can be expected. (example: Al, Cu, V) (Bensadok, Kenza & Benammar, Souad & Lapicque, François & Nezzal, G. (2008). Journal of hazardous materials. 152. 423-30; Gao, Feng & Olayiwola, Afolabi & Liu, Biao & Wang, Shaona & Du, Hao & Li, Jianzhong & Wang, Xindong & Chen, Donghui & Zhang, yi. (2021). Review of Vanadium Production Part I: Primary Resources. Mineral Processing and Extractive Metallurgy Review. 43. 1-23).

In the specific case of MB, complexes with the metal form of precious metals (most likely nanoparticles) can be favoured due to the availability of the S doublet (divalent sulphur). That includes affinity for Ag, or Au and potentially other thiophilic compounds.

It is likely, based on the process disclosed herein, that the formation of the complex only favours the intake of both the photosensitisers and the metal particles in the medium to be loaded (most often polymer). It is expected that upon returning to neutral pH (PBS washes) the complex would be broken and that the metal particles would return to their solid/insoluble state.

The beneficial effect associated can be attributed two 2 factors:
1. The higher intake of both species by the final medium, leading to a better efficiency of their antibacterial properties.
2. The conjugated effect (synergy) of the 2 antibacterial agents, most likely relying on different mechanism of action, the photosensitisers through ROS (oxygen radicals) formation leading to cell/DNA oxidation, the metal nanoparticles through membrane accumulation/permeation leading to apoptosis (Bruna T, Maldonado-Bravo F, Jara P, Caro N. Int J Mol Sci. 2021 Jul. 4; 22(13): 7202).

### 4. Mechanisms of Reactivity of Methylene Blue in the Dark

In the presence of light, photons with specific energy level can activate MB. Once activated MB can react with “classic” oxygen also called triplet oxygen (³O₂) to form singlet oxygen (¹O₂). The difference of these 2 types of oxygen lies in the outer electron shell configuration.

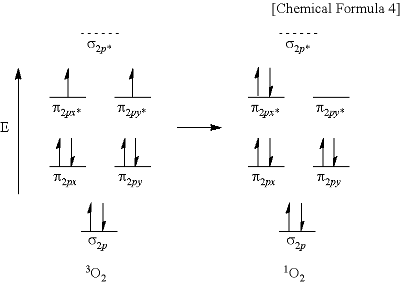

Having an empty orbital results in increased reactivity of singlet oxygen to facilitate oxidation steps, such as the oxidation of lipids or proteins that constitute the bacterial cell wall and lead to their destruction.

In this case, MB acts as a catalyst and is regenerated under its oxidated form (blue).

In the example below, we can see the oxidation by singlet oxygen of a C—C bond in dioxethane leading to its separation in 2 units along with the catalytic cycle of MB (Lee, J. Bioluminescence, the Nature of the Light, (2020) University of Georgia).

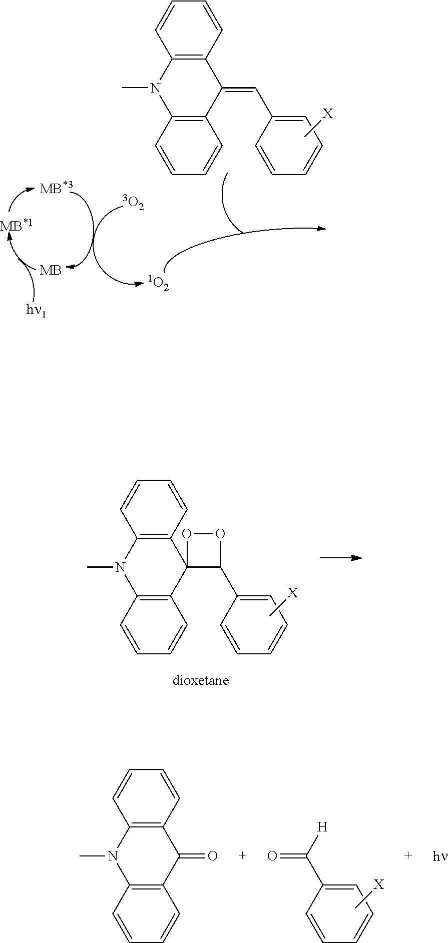

In the dark, the absence of photons means there is no basis for the catalytic cycle of the MB and therefore no formation of singlet oxygen. However, in basic or acid conditions the reaction of the MB in its oxidized form (blue) with organic species (such as bacteria cell wall) is catalyzed, the source of electrons and protons being the bacteria itself. This reaction, however, is not reversible, and will progressively lead to the reduction of the MB in leucomethylene blue (LMB) (reduced form, colorless) (Mahlum, J D et al., J. Phys. Chem. C 2021, 125, 17, 9038-9049).

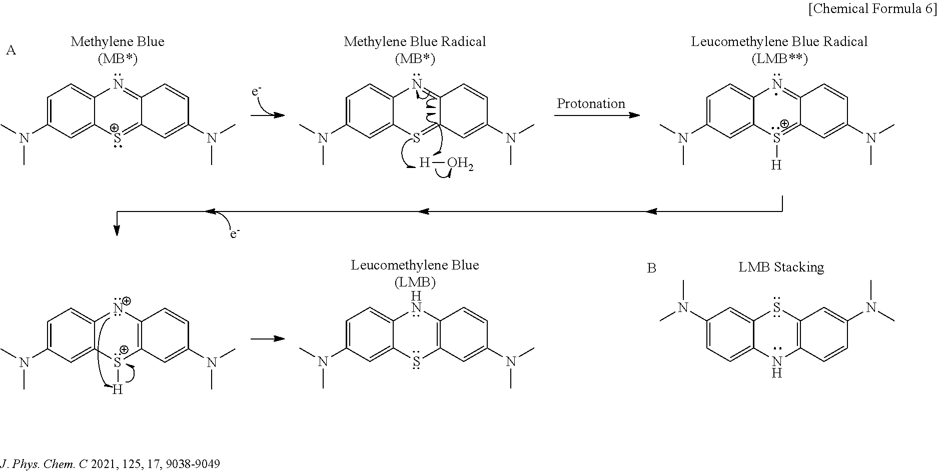

In the invention provided herein, we can expect the presence of zincate (in the form of a MB-zincate complex) to act as the catalyst as a basic source (equilibrium zincate⇔zinc oxide+hydroxide ion).

It is expected, according to this theory, that the MB would progressively become inactive as it is reduced, but this is not observed visually (the reduction from blue to colorless) in practice when samples are exposed to bacteria in light conditions. Depending on the concentration encapsulated, the time to inactivity could vary (based on flow rate and exposure to reducing agents, such as bacteria). As such high concentrations are encapsulated, it's expected this is why this reduction in activity has not been observed in experiments. However, there is a possibility that dissolved oxygen could lead to the oxidation of LMB even without light activation. This can be observed in the famous blue bottle experiment (The ‘blue bottle’ experiment in association with Nuffield Foundation).

### II. Methods of Making

### A. Process for Preparation of the Antimicrobial Conjugates

In some embodiments, the antimicrobial conjugates are in the form of a solution. Such a solution may be produced by contacting a solution of charge-stabilized metallic nanoparticles with a solution of one or more photosensitizers. The mixtures are contacted at any suitable temperature, for example between the freezing point and boiling point of the solvent employed (or at a temperature at which both solutions are liquid if different solvents are employed). However, if the temperature is too high, the nanoparticle solution may become unstable. In some embodiments, the solutions are contacted at or about room temperature.

In some embodiments, a solution of metallic nanoparticles is mixed with a solution of one or more photosensitizers and swell encapsulated into a polymer. In some embodiments, the solution containing the metallic nanoparticle and photosensitizer mixture is allowed to stand at room temperature for about 10 minutes, about 10 minutes to about 1 hour, or about 15 minutes to about 20 minutes.

In some embodiments, the metallic nanoparticle solution and/or the photosensitizer solution is a solution in a polar solvent. In some embodiments, the polar solvent is an aqueous solution, such as water or phosphate buffered saline solution, in a pharmaceutically acceptable aqueous carrier. In some embodiments, both the nanoparticle and photosensitizer solutions are aqueous.

The pH of solutions may be such that no adjustment is required upon mixing, or the pH of the mixture may be controlled by the use of a suitable buffer. For example, when the mixture is to be applied to the body, the pH of the mixture should not be outside the physiological pH range for the site. The physiological pH range depends on the site in question, e.g. intact skin can have a pH as low as 4.2. In some embodiments, the pH of the solutions described herein are between pH 5.5 and 8.0. In other embodiments, the pH of the solutions described herein are between pH 3.0 and 5.0.

The two solutions may be mixed in any proportion, such that the desired concentration is achieved in the mixed solution. In one embodiment, the initial concentrations of each solution are selected as required so that the desired concentration in the mixed solution is achieved when equal volumes of metallic nanoparticle solution and photosensitizer solution are mixed together.

The desired concentration of the nanoparticles in the mixture depends on the desired final concentration at the site to be treated. This may vary and a suitable choice depends both on the size of the nanoparticle and the concentration of the photosensitizer solution. In some embodiments, the final concentration of the nanoparticles in the mixture is from about 1×10¹¹ to about 5×10¹⁵ particles/ml. In some embodiments, the final concentration is from about 3×10¹¹ to about 1×10¹⁵ particles/ml. In order to obtain such a final concentration, the initial concentration of the nanoparticle solution is from about 1×10¹² to about 1×10¹⁶ particles/ml. If the nanoparticle solution as prepared, or as obtained commercially, is of higher concentration than this, it may be necessary to dilute the nanoparticle solution before mixing with the photosensitizer. For example, an original nanoparticle solution containing 1×10¹⁴ or 1×10¹⁵ particles/ml may be diluted 1:10 to 1:100, such that the concentration before mixing with the photosensitizer solution is from 1×10¹² to 1×10¹⁴.

In various embodiments, the initial concentration of photosensitizer solution is chosen such that when mixed with the nanoparticle solution, the final concentration of photosensitizer at the treatment site is from about 500 to about 600 mg/L, 600 to about 700 mg/L, 700 to about 800 mg/L, 800 to about 900 mg/L more preferably from about 800 mg/L.

It should be noted that the final concentration at the treatment site may not necessarily correspond to the concentration in the mixed solution. For instance in the treatment of periodontal pockets and wounds the treatment site may be flooded with body fluid such as saliva or blood. In such cases, it may therefore be necessary to apply the nanoparticle-photosensitizer mixture in greater concentration so as to achieve an effective concentration after dilution by the body fluid.

### B. Swell Encapsulation of Polymers

The methods provided herein are for swell encapsulation using already commercially available polymers used in medical devices (tends to be softer polymers) such as drains, catheters, endotracheal tubes, sutures etc. Some examples of the polymers used include, but are not limited to latex used in penrose drains, PVC/PDMS/Silicone/PU used in catheters and endotracheal tubes, PGA/PLGA/polytetrafluoroethylene nylon, poly(ethylene terephthalate), polypropylene polybutester, and poly(vinylidene fluoride) used in sutures.

The swell encapsulation method used is mostly as described in U.S. Pat. No. 8,580,309B2 (hereby incorporated by reference). However, as a result of unsuccessful attempts to swell encapsulate the zinc oxide and methylene blue compositions into polymers using the methods of U.S. Pat. No. 8,580,309B2, the inventors discovered that the conditions at which the swell encapsulation was done are critical. This invention discloses that the addition of a base such as sodium hydroxide (NaOH), which is not disclosed in U.S. Pat. No. 8,580,309B2, allows the zinc oxide nanopowder to become soluble in this water/acetone mix, and therefore able to swell encapsulate. Additionally, in basic conditions, Zinc oxide (ZnO) will react to form sodium zincate. Compared to ZnO, zincate ions are very soluble in water which led to much easier swell encapsulation in water-based solutions.

In particular, the method provided herein comprises encapsulating a polymer with the antimicrobial compositions provided herein. The method comprises dissolving the metallic nanoparticle and photosensitizer dye in a solvent mixture. In some embodiments, the solvent mixture comprises a water/acetone mixture. In various embodiments, the water/acetone mix is at a ratio of 99:1, 90:10, 70:30, 60:40, or 50:50 of water to acetone. The method further comprises adding a pH agent to the metallic nanoparticle and photosensitizer dye mixture solution based on the v/v % of the pH agent. In some embodiments, about 0.1%-10% of NaOH is added to the metallic nanoparticle and photosensitizer dye mixture solution based on the v/v % of the pH agent based on the v/v % of NaOH. In some embodiments, about 2% of NaOH is added to the metallic nanoparticle and photosensitizer dye mixture solution based on the v/v % of the pH agent. The polymer is then placed into metallic nanoparticle and photosensitizer dye mixture solution so that there is enough solution to cover the polymer and incubated at room temperature away from direct light for about 8 to 48 hours. In some embodiments, the polymer is incubated at room temperature in dark conditions from about 8 to 16 hours, 12 to 20 hours, 16 to 24 hours, 20 to 28 hours, 24 to 32 hours, 28 to 36 hours, 32 to 40 hours, 36 to 44 hours, and/or 40 to 48 hours.

In some embodiments, the base is selected from any base that is used to change the pH of a solution such as NaOH, sodium bicarbonate, potassium hydrogen carbonate, potassium hydroxide etc. In some embodiments, adding a few drops of >0.1% NaOH to the solution to bring the pH to about 7 provides dark blue sample, indicating that both the polymer was successfully swell encapsulated with the antimicrobial components. In some embodiments, the pH is between 6 and 7. Attempts to swell encapsulate methylene blue on its own at the same concentration resulted in very light colored material, indicating that that the polymer was not successfully swell-encapsulated with methylene blue alone. The success of using NaOH also allowed reduction the amount of acetone used and increased the amount of water in the solvent mix, which is better for the integrity of the polymer.

In some embodiments, methylene blue and ZnO antimicrobial compositions are added directly with polymer prior to it going through the extrusion process. Once the polymer is in pellet form or being melted down, prior to extrusion, MB and ZnO are mixed in with the melted pellet (preferably as late as possible to reduce exposure to heat) then the polymer is extruded directly with the MB and ZnO mixed in. This is how many medical devices are made such as sutures, catheters, endotracheal tubes etc., so this would be hugely advantageous over swell encapsulation methods which occur after the manufacturing process.

In other embodiments, the methylene blue and ZnO antimicrobial compositions may be added to the polymer before extrusion or after extrusion. The polymers may include but are not limited to polymers used in medical devices such as penrose drains (latex), feeding tubes (PVC), urinary catheters (PVC), endotracheal tubes (PVC), suture imitation* (polyamide or nylon 6 tubing), endotracheal tubes (siliconized PVC), winged infusion set (PVC), catheters (polyurethane) and devices made of silicone and other polymers.

In other embodiments, the methylene blue and ZnO antimicrobial compositions may be added to metals or polymers during casting, machining, 3-D printing, or other processes to form implantable devices such as orthopedic implants. The metals or polymers may include but are not limited to metals or polymers used in medical devices such stainless steel, cobalt-based alloys, titanium, polycaprolactone (PCL), polylactide (PLA), and polyglycolide (PGA).

### III. Methods of Treatment

### A. Antimicrobial Effect of the Mixtures

The mixtures of the present invention have an antimicrobial effect, i.e. they are capable of killing or inhibiting the growth of microorganisms, including bacteria, viruses, fungi and prions, that can cause disease in humans, animals or plants. In some embodiments, the mixtures of the present invention are used to kill or inhibit the growth of Staphylococcus aureus. Staphylococcus aureus as used in this application shall also include Methicillin-Resistant Staphylococcus aureus (“MRSA”). The mixtures of the present invention may also be used to kill or inhibit the growth of Propionibacterium acnes.

In other embodiments, the mixtures of the present invention are used to kill or prevent the growth of the microbes involved in in wound infections and in disinfecting or sterilizing wounds and other lesions on a subject. Thus, the mixtures of the present invention may be used to kill or inhibit the growth of Streptococcus sanguis, Porphyromonas gingivalis, Fusobacterium nulceatum, Actinobacillus actinomycetemcomitans, Candida albicans, Streptococcus mutans and lactobacilli, Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis Bacteroides gingivalis, Bacteroides melaninogenicus (also known as Prevotella melaninogenica), Bartonella henselae, Bartonella quintana, Bordetella bronchiseptica Bordetella pertussis, Borrelia burgdorferi, Brucella abortus, Brucella melitensis, Brucella suis, Burkholderia, Burkholderia mallei, Burkholderia pseudomallei, Burkholderia cepacian, Calymmatobacterium granulomatis, Campylobacter coli, Campylobacter fetus, Campylobacter jejuni, Helicobacter pylori, Chlamydia trachomatis, Chlamydophila, Chlamydophila pneumoniae (also called Chlamydia pneumoniae), Chlamydophila psittaci (also called Chlamydia psittaci), Clostridium botulinum, Clostridium difficile, Clostridium perfringens (also called Clostridium welchii), Clostridium tetani, Corynebacterium, Corynebacterium diphtheriae, Corynebacterium fusiforme, Coxiella burnetiid, Ehrlichia chaffeensis, Ehrlichia ewingii, Fikenella corrodens, Enterobacter 17 5 10 15 20 25 30 35 cloacae, Enterococcus avium, Enterococcus durans, Enterococcus faecalis, Enterococcus faecium, Enterococcus gallinarum, Enterococcus maloratus, Escherichia coli, Fusobacterium necrophoru, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leptospira interrogans, Leptospira noguchii, Listeria monocytogenes, Methanobacterium extroquens, Microbacterium multiforme, Micrococcus luteus, Moraxella catarrhalis Mycobacterium, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Peptostreptococcus, Porphyromonas gingivalis, Prevotella melaninogenica (previously called Bacteroides melaninogenicus), Pseudomonas aeruginosa, Rhizobium radiobacter, Rickettsia prowazekii, Rickettsia psittaci, Rickettsia quintana, Rickettsia rickettsia, Rickettsia trachomae, Rochalimaea, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia Streptococcus agalactiae, Streptococcus avium, Streptococcus bovis, Streptococcus cricetus, Enterococcus faecium, Enterococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Ureaplasma urealyticum, Vibrio cholerae, Vibrio comma, Vibrio parahaemolyticus, Vibrio vulnificus, Wolbachia, Yersinia enterocolitica, Yersinia pestis, Yersinia pseudotuberculosis The bacteria may be selected from Acinetobacter baumannii, Pseudomonas aeruginosa, Enterobacteriaceae, Enterococcus faecium, Staphylococcus aureus, Helicobacter pylori, Campylobacter spp, Salmonellae, Neisseria gonorrhoeae, Haemophilus influenzae, Shigella spp. The bacteria may be a bacteria resistant to one or more antibiotics, for example a bacterium selected from Acinetobacter baumannii (carbapenem-resistant), 18 Pseudomonas aeruginosa (carbapenem-resistant), Enterobacteriaceae (carbapenem resistant, ESBL-producing), 5 Enterococcus faecium (vancomycin-resistant), Staphylococcus aureus (methicillin-resistant and or vancomycin-intermediate and resistant), Helicobacter pylori (clarithromycin-resistant), Campylobacter spp., (fluoroquinolone-resistant), Salmonellae (fluoroquinolone resistant), Neisseria gonorrhoeae (cephalosporin-resistant and or fluoroquinolone resistant), Streptococcus pneumoniae (penicillin-non-susceptible), Haemophilus influenzae (ampicillin-resistant), Shigella spp., (fluoroquinolone-resistant).

### B. Applications of Mixtures

The invention provides the use of the compositions of the present invention in the manufacture of a medicament for use in disinfecting or sterilizing tissues of a body cavity or a wound or lesion in a body cavity by contacting the tissues, wound or lesion with mixture.

The wound or lesion treated may be any surgical or trauma-induced wound, a lesion caused by a disease-related microbe, or a wound or lesion infected with such a microbe. The treatment may be applied to disinfect or sterilize a wound or lesion as a routine precaution against infection or as a specific treatment of an already diagnosed infection of a wound or lesion. In some embodiments, the body cavity is the oral cavity. The mixtures of the present invention may also be used in other body cavities, such as the nose, rectum, vagina, etc. In other embodiments, the compositions of the present invention may be used on devices to be implanted within a subject's body.

In another aspect the invention provides the use of a mixture of the present invention in the manufacture of a medicament for use in killing or preventing the growth of disease-related microbes in any body orifices or any body cavity such as the oral cavity, nose, rectum, vagina, ear, eyes, inside any incision/wound/cut etc, or on implanted devices by contacting the microbes with mixture.

For the above applications, the mixture is suitably used in the form of a pharmaceutical composition comprising the nanoparticles and photosensitizer in solution in a pharmaceutically acceptable aqueous carrier. The pharmaceutical composition may further comprise one or more accessory ingredients selected from buffers, salts for adjusting the tonicity of the solution, antioxidants, preservatives, gelling agents and remineralization agents.

As described above, the mixture is at a suitable concentration such that a desired level of antimicrobial activity is achieved at the treatment site.

The mixture may be left in contact with the microbes for a period of time. This duration of time may vary depending on the particular photosensitizer in use and the target microbes to be killed. Time of contact will entirely depend on the best course of action for the device (e.g. hours, days to weeks). The mixture will start being antimicrobial instantly and has been shown to reach the >99.99% reduction in under 3 hours. In some embodiments, the duration of time of contact can be from about 1 second to about 10 minutes. In some embodiments, the duration of time is about 10 minutes to about 1 hour. In other embodiments, the duration of time is about 1 hour to about 3 hours. In yet other embodiments, the duration of time is about 3 hours to about 5 hours. In other embodiments, the antimicrobial mixture is left in the body cavity indefinitely.

### EXAMPLES

### Example 1: Antimicrobial Activity of a Wound Drain With Zinc Oxide and Methylene Blue Antimicrobial Compositions

### Materials and Methods

A penrose wound drain is swell encapsulated with zinc oxide and methylene blue antimicrobial mix. This process involves dissolving the nanoparticle and photosensitizer dye in a water/acetone mix. The water to acetone ratios may vary from a 50:50 ratio to a 99:1 ratio. In one experiment, a 70 water:30 acetone ratio is used to reach a 800 mg/L concentration of nanoparticles/photosensitizer in the solution, with the addition of about 2% volume of 0.1M NaOH, solublizing the zinc oxide. The polymer is then placed into this solution ensuring it's completely covered and left overnight at room temperature in dark conditions. In this particular experiment, the polymer solution was placed on a roller to provide continuous mixing. Then, the polymer is washed at 45 degrees Celsius for 5 minutes in PBS. This is repeated with fresh PBS 5 times at 40° C. in sterile PBS. Then, the polymer is washed at room temperature for 5 minutes in sterile water. This is repeated with fresh sterile water 5 times at room temperature (total 10 washes). The polymer is then allowed to dry.

### Results

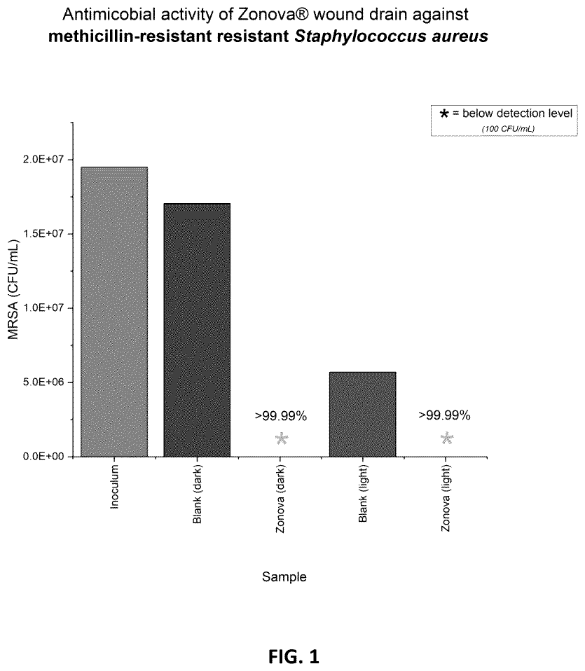

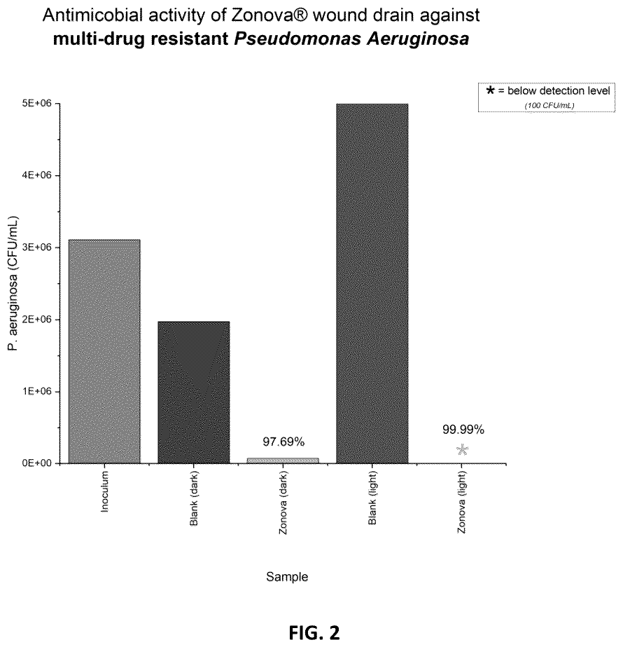

FIGS. 1 and 2 show that when a wound drain was swell encapsulated with antimicrobial compositions, antimicrobial activity was seen against methicillin-resistant Staphylococcus aureus and multi-drug resistant Pseudomonas aeruginosa. The use of zinc oxide allows activity in the dark, which is important for using the antimicrobial composition in body cavities where light is not available to activate the photosensitizer.

The pH change bought about by the addition of the sodium hydroxide allows the zinc oxide nanopowder to become soluble in this water/acetone mix, and therefore able to swell encapsulate. Adding about 0.1 to 10% NaOH to the solution based on the v/v % of NaOH provided material resulted in a very dark blue in color, indicating that both the polymer was successfully swell encapsulated with the antimicrobial components In some embodiments, about 2 to 5% of NaOH is added to the solution based on the v/v % of NaOH. In some embodiments, about 2% of NaOH is added to the solution based on the v/v % of NaOH. Attempts to swell encapsulate methylene blue on its own at the same concentration resulted in very light-colored material, indicating that that the polymer was not successfully swell-encapsulated with methylene blue alone. The success of using NaOH also allowed reduction the amount of acetone used, and increased the amount of water in the solvent mix, decreasing the risk of altering the integrity of the polymer.

Studies were conducted to the optimal pH for swell encapsulations. Experiments were conducted with 50/50, 70/30, 90/10 and 99:1 DIW/Acetone mixes adding NaOH in 100 uL amounts incrementally. In all cases, the pH of solution was >14. When dye was added, it becomes too dark to see. This indicated that there may be some synergies between all components at play where the components are dependent on each other for successful swell encapsulation.

FIG. 3 is pH study in a 70/30 DIW/Acetone mix with ZnO/dye and polymer samples inside each, with incremental amounts of NaOH in each solution. The data illustrate that any addition of NaOH increases the swell encapsulation capabilities of methylene blue and ZnO; however, with rose Bengal and ZnO the same doesn't apply.

### Example 2: Antimicrobial Activity of Rose Bengal Compositions

It was initially assumed that conditions favorable to MB would also be favorable to Rose Bengal (RB), another photosensitizer that also acts as a very good antimicrobial, showing the same high level of antimicrobial activity as methylene blue when used in conjunction with ZnO in polymers. However, the experiment presented in FIG. 3 (using latex penrose drain pieces) shows that pH has opposite effect on MB and RB.

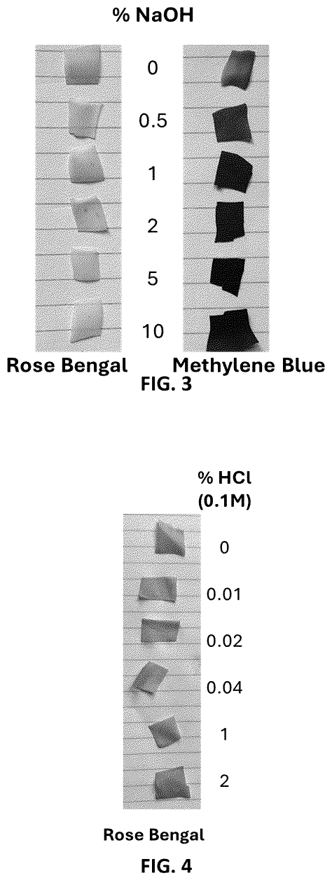

It has been reported that acidic conditions are more favorable for Rose Bengal (RB) absorption and that basic conditions tend to promote release of RB (Gupta, V K et al., RSC Adv., 2012, 2, 8381-8389). Based on these findings, we attempted swell encapsulation of RB in acid conditions using HCl as an acid source, which confirmed that the addition of an acidic compound improved swell encapsulation (FIG. 4). It is unclear based on current data how the use of HCl would impact the encapsulation process of the ZnO particles. The following reaction is usually expected mostly in concentrated condition:

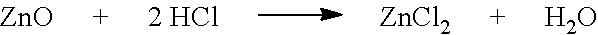

The transformation to ZnCl₂ would be extremely beneficial to solubility in water and could form a complex with the negatively charged RB ion [RB⁻]-[Zn²⁺]-[RB⁻]:

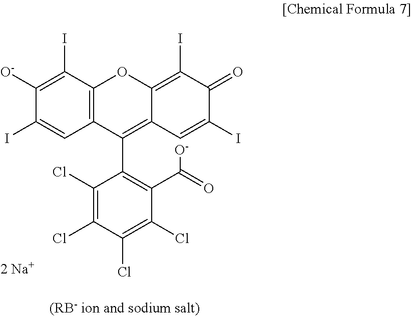 [SMILES: O=C([O-])c1c(Cl)c(Cl)c(Cl)c(Cl)c1-c1c2cc(I)c(=O)c(I)c-2oc2c(I)c([O-])c(I)cc12]

### Example 3: Swell Encapsulation of Methylene Blue and ZnO Into Implantable Medical Devices

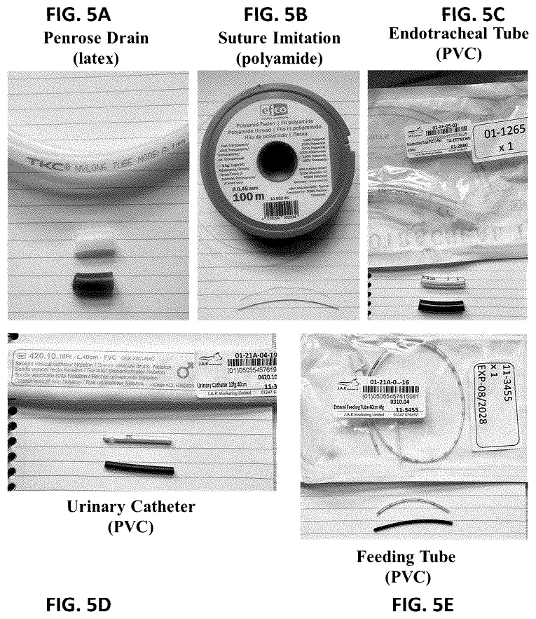

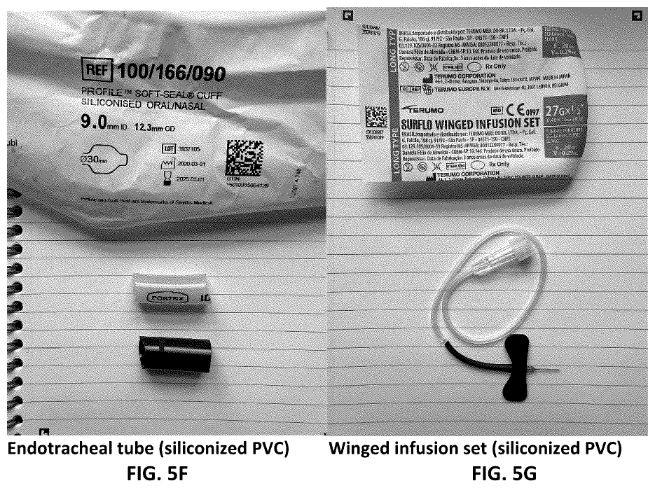

FIGS. 5A-5E show that multiple implantable medical devices including penrose drains—latex (FIG. 5A), suture imitation—polyamide (FIG. 5B), endotracheal tubes—PVC (FIG. 5C), urinary catheters—PVC (FIG. 5D), and feeding tubes—PVC (FIG. 5E) were successfully swell encapsulated with the methylene blue and ZnO antimicrobial mixtures. This data shows that a range of medical device polymers may undergo swell encapsulation with the antimicrobial mixtures disclosed herein.

### Example 4: Antimicrobial Surface Test in Both Light and Dark Conditions

Following procedures laid out in Examples 1 and 2, the antimicrobial properties of various materials were tested against E. coli and S. epidermis under both dark and light conditions, both with 9 hour exposure time, with the conditions noted in Tables 1-4

E. coli Summary of Results

TABLE 1
Dark conditions with 9 hour exposure time:

| Entry No. | Device | Polymer | Dye | NP | Solvent mix | Bacteria Tested | % Reduction |
|---|---|---|---|---|---|---|---|
| 1. | ET tube | PVC | — | — | — | E. coli | 0.000 |
| 2. | Urinary | PVC | MB | ZnO | DIW/Acetone | E. coli | 72.791 |
| | Catheter | | | | 70/30, | | |
| | | | | | 5% NaOH 0.1M | | |
| 3. | ET tube | PVC | MB | Cu | DIW/MEK | E. coli | 99.999 |
| | | | | | 70/30, | | |
| | | | | | 5% NaOH 0.1M | | |
| 4. | Drain | Latex | — | — | — | E. coli | 0.000 |
| 5. | Drain | Latex | MB | ZnO | DIW/Acetone | E. coli | 19.565 |
| | | | | | 70/30, | | |
| | | | | | 5% NaOH 0.1M | | |
| 6. | Drain | Latex | MB | Cu | DIW/MEK | E. coli | 97.174 |
| | | | | | 70/30, | | |
| | | | | | 5% NaOH 0.1M | | |

TABLE 2
Light conditions (~3,000 lux) with 9 hour exposure time:

| Entry No. | Device | Polymer | Dye | NP | Solvent mix | Bacteria Tested | % Reduction |
|---|---|---|---|---|---|---|---|
| 1. | ET tube | PVC | — | — | — | E. coli | 0.000 |
| 2. | Urinary | PVC | MB | ZnO | DIW/Acetone | E. coli | 95.349 |
| | Catheter | | | | 70/30, | | |
| | | | | | 5% NaOH 0.1M | | |
| 3. | ET tube | PVC | MB | Cu | DIW/MEK | E. coli | 99.999 |
| | | | | | 70/30, | | |
| | | | | | 5% NaOH 0.1M | | |
| 4. | Drain | Latex | — | — | — | E. coli | 0.000 |
| 5. | Drain | Latex | MB | ZnO | DIW/Acetone | E. coli | 99.783 |
| | | | | | 70/30, | | |
| | | | | | 5% NaOH 0.1M | | |
| 6. | Drain | Latex | MB | Cu | DIW/MEK | E. coli | 99.999 |
| | | | | | 70/30, | | |
| | | | | | 5% NaOH 0.1M | | |

S. epidermis Summary of Results

TABLE 3
Dark conditions with 9 hour exposure time:

| Entry No. | Device | Polymer | Dye | NP | Solvent mix | Bacteria Tested | % Reduction |
|---|---|---|---|---|---|---|---|
| 1. | ET tube | PVC | — | — | — | S. epidermis | 0.000 |
| | control | | | | | | |
| 2. | Urinary | PVC | MB | ZnO | DIW/Acetone | S. epidermis | 35.714 |
| | Catheter | | | | 70/30, | | |
| | | | | | 5% NaOH | | |
| | | | | | 0.1M | | |
| 3. | ET tube | PVC | MB | Cu | DIW/MEK | S. epidermis | 99.999 |
| | | | | | 70/30, | | |
| | | | | | 5% NaOH | | |
| | | | | | 0.1M | | |
| 4. | Drain | Latex | — | — | — | S. epidermis | 0.000 |
| | control | | | | | | |
| 5. | Drain | Latex | MB | ZnO | DIW/Acetone | S. epidermis | 91.000 |
| | | | | | 70/30, | | |
| | | | | | 5% NaOH | | |
| | | | | | 0.1M | | |
| 6. | Drain | Latex | MB | Cu | DIW/MEK | S. epidermis | 99.9400 |
| | | | | | 70/30, | | |
| | | | | | 5% NaOH | | |
| | | | | | 0.1M | | |

TABLE 4
Light conditions (~3,000 lux) with 9 hour exposure time:

| Entry No. | Device | Polymer | Dye | NP | Solvent mix | Bacteria Tested | % Reduction |
|---|---|---|---|---|---|---|---|
| 1. | ET tube | PVC | — | — | — | S. epidermis | 0.000 |
| | control | | | | | | |
| 2. | Urinary | PVC | MB | ZnO | DIW/Acetone | S. epidermis | 71.429 |
| | Catheter | | | | 70/30, | | |
| | | | | | 5% NaOH | | |
| | | | | | 0.1M | | |
| 3. | ET tube | PVC | MB | Cu | DIW/MEK | S. epidermis | 99.999 |
| | | | | | 70/30, | | |
| | | | | | 5% NaOH | | |
| | | | | | 0.1M | | |
| 4. | Drain | Latex | — | — | — | S. epidermis | 0.000 |
| | control | | | | | | |
| 5. | Drain | Latex | MB | ZnO | DIW/Acetone | S. epidermis | 99.998 |
| | | | | | 70/30, | | |
| | | | | | 5% NaOH | | |
| | | | | | 0.1M | | |
| 6. | Drain | Latex | MB | Cu | DIW/MEK | S. epidermis | 99.998 |
| | | | | | 70/30, | | |
| | | | | | 5% NaOH | | |
| | | | | | 0.1M | | |

### Example 5: Swell Encapsulation Key Parameters

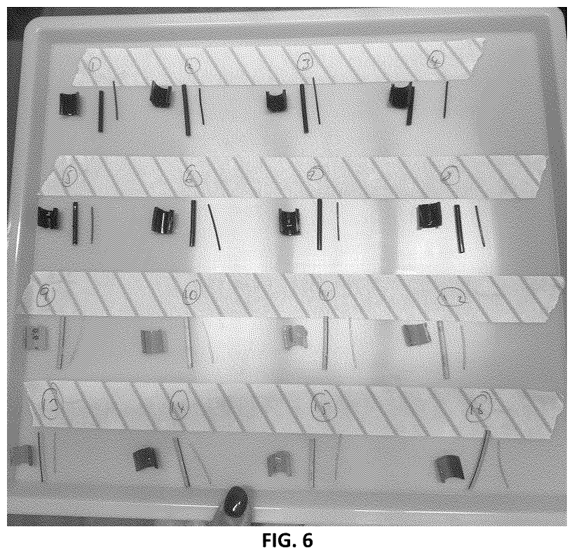

Following procedures laid out in Examples 1 and 2, the effect of various factors was tested on the swell encapsulation of three polymers: polyurethane Tom Tiddle urinary catheter, 100% silicone Anifoley urinary catheter, and siliconised PVC Smiths Portex ET tube. These experiments are presented in Table 5, and results are depicted in FIG. 6. These results suggest that different polymers prefer different mixes (e.g. between 1 and 2, the addition of 30% acetone to the swelling mixture increased the darkness of both the PVC and polyurethane polymer samples, indicating successful swell encapsulation). Copper samples did not swell well in 100% water specifically for the polyurethane polymer, swelling better in the 70/30 water/acetone mix. PVC and siliconized polymers were not so specific. The combinations used with RB did not swell as well, with 70/30 water/acetone and Cu as the chosen nanoparticle for the PVC, polyurethane, and siliconized samples providing the best results.

The results of experiments run at a concentration of 50 mg/L and 1% NaOH still yielded coloured polymers, indicating swell encapsulation had occurred, but they were lighter (suggesting less antimicrobial efficacy). Experiments with 100 mg/L concentration and 1% NaOH yielded similar results in terms of darkness found in samples which were swell encapsulated at 800 mg/L at 5%. For the polymer types tested, 100 mg/L at 1% is the minimum concentration required for optimal antimicrobial activity for most polymers, but this is not necessarily a limiting parameter for other types of polymers listed and under other conditions.

TABLE 5
Swell encapsulation parameters.

| | | | Concentration | Solvent | pH adjustment |
|---|---|---|---|---|---|
| Run | Photosensitiser | Nanoparticle | (mg/L) | ratio | (5%) |
| 1 | MB | ZnO | 200 | 100 water | NaOH |
| 2 | MB | ZnO | 200 | 70/30 W/A | NaOH |
| 3 | MB | ZnO | 800 | 100 water | NaOH |
| 4 | MB | ZnO | 800 | 70/30 W/A | NaOH |
| 5 | MB | Cu | 200 | 100 water | NaOH |
| 6 | MB | Cu | 200 | 70/30 W/A | NaOH |
| 7 | MB | Cu | 800 | 100 water | NaOH |
| 8 | MB | Cu | 800 | 70/30 W/A | NaOH |
| 9 | RB | ZnO | 200 | 100 water | HCl |
| 10 | RB | ZnO | 200 | 70/30 W/A | HCl |
| 11 | RB | ZnO | 800 | 100 water | HCl |
| 12 | RB | ZnO | 800 | 70/30 W/A | HCl |
| 13 | RB | Cu | 200 | 100 water | HCl |
| 14 | RB | Cu | 200 | 70/30 W/A | HCl |
| 15 | RB | Cu | 800 | 100 water | HCl |
| 16 | RB | Cu | 800 | 70/30 W/A | HCl |

### Example 6: Solvent Experiment With POBE and Latex Polymers

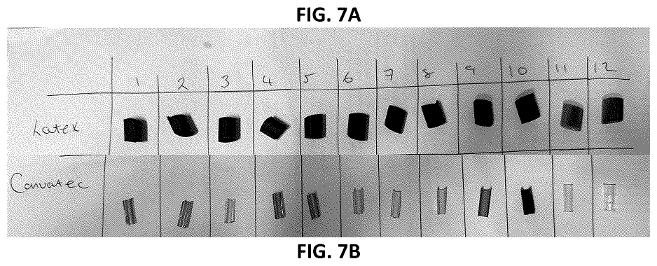

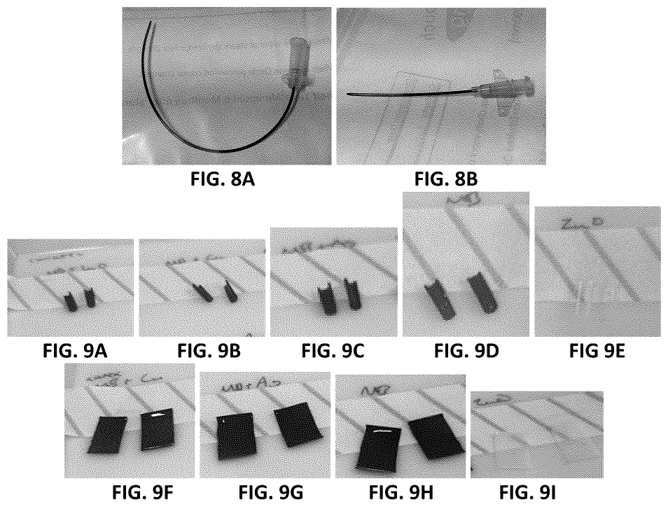

According to the method presented in Experiment 1, a polyolefin-based clastomer (POBE) polymer urinary catheter was tested using MB and ZnO (800 mg/L) with different solvent mixtures and compared to a latex drain (Table 6). Latex swells easily in multiple solvents, aside from a water/ethyl acetate mixture (FIG. 7A). The POBE catheter swells only in 70/30 water/methyl ethyl ketone (MEK) (FIG. 7B).

TABLE 6
Solvent experiment.

| | | | pH | Y/N | Y/N Convatec |
|---|---|---|---|---|---|
| | | | (0.1M | Drain | catheter |
| Solvent | Run | Ratio | NaOH) | (Latex) | (POBE) |
| Acetone/water | 1 | 50/50 | 5% | Y | ~ |
| | 2 | 70/30 | 5% | Y | ~ |
| | 3 | 100 | 5% | Y | N |
| Water/acetone | 4 | 50/50 | 5% | Y | ~ |
| | 5 | 70/30 | 5% | Y | ~ |
| | 6 | 100 | 5% | Y | N |
| Water/IPA | 7 | 50/50 | 5% | Y | N |
| | 8 | 70/30 | 5% | Y | N |
| water/MEK | 9 | 50/50 | 5% | Y | ~ |
| | 10 | 70/30 | 5% | Y | Y |
| water/Ethyl acetate | 11 | 50/50 | 5% | N | N |
| | 12 | 70/30 | 5% | N | N |

### Experiment 7: Antimicrobial Testing Results With Various Polymers, Dyes, and Nanoparticles

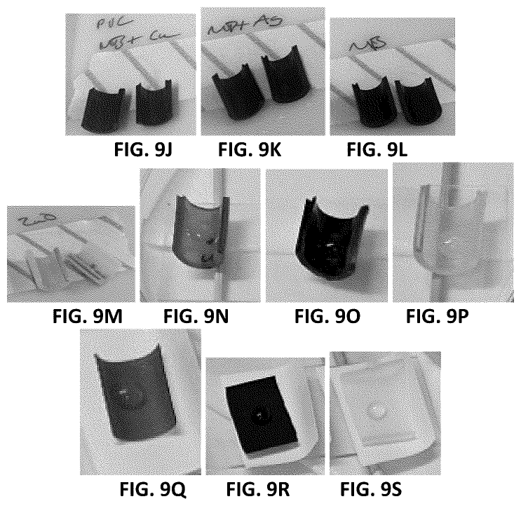

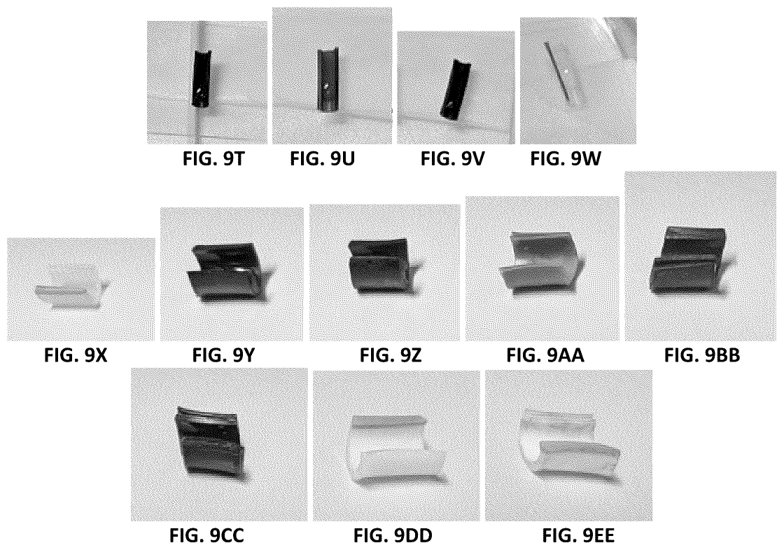

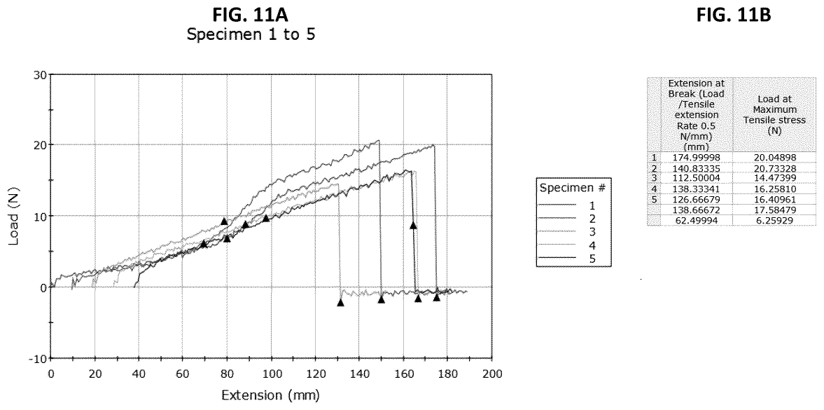

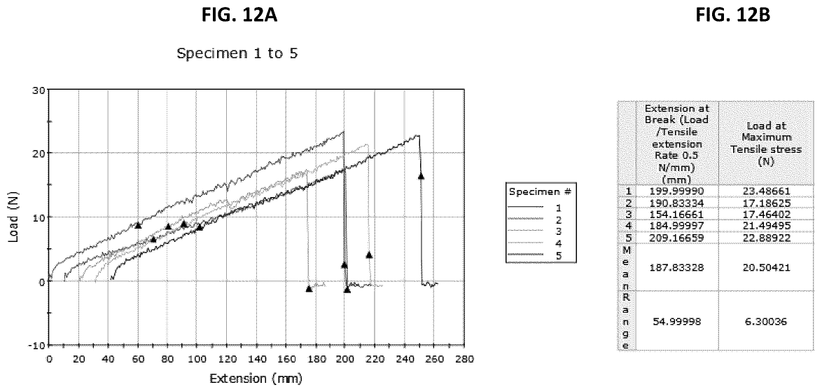

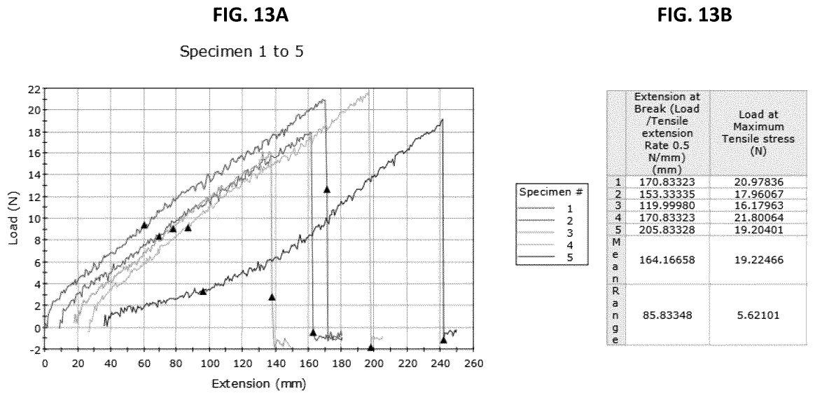

According to the method presented in Experiment 1, a variety of polymers, dyes, and nanoparticles were tested across a range of bacteria. Notably, ET-Tube PVC based samples showed >90% antimicrobial activity across a range of dye and nanoparticle combinations (Table 7). Latex-based drain samples showed >99% antimicrobial activity for all dye and nanoparticle combinations tested. POBE-based urinary catheter samples showed >90% antimicrobial activity across a range of dye and nanoparticle combinations (FIG. 9A-EE).

TABLE 7
Antimicrobial testing.

| Polymer | Sample Type | Dye | NP | Solvent mix | Bacteria Tested | % Reduction |
|---|---|---|---|---|---|---|
| PVC | Endotracheal | MB | Ag | DIW/Acetone | E. coli | 99.48 |
| | tube | | | 70/30, | | |
| | | | | 5% NaOH | | |
| | | | | 0.1M | | |
| PVC | Endotracheal | MB | Cu | DIW/Acetone | E. coli | 99.52 |
| | tube | | | 70/30, | | |
| | | | | 5% NaOH | | |
| | | | | 0.1M | | |
| PVC | Endotracheal | MB | — | DIW/Acetone | E. coli | 99.61 |
| | tube | | | 70/30, | | |
| | | | | 5% NaOH | | |
| | | | | 0.1M | | |
| PVC | Endotracheal | — | ZnO | DIW/Acetone | E. coli | 3.23 |
| | tube | | | 70/30, | | |
| | | | | 5% NaOH | | |
| | | | | 0.1M | | |
| PVC | Endotracheal | MB | Cu | DIW/MEK | E. coli | 99.999 |
| | tube | | | 70/30, | | |
| | | | | 5% NaOH | | |
| | | | | 0.1M | | |
| PVC | Endotracheal | RB | ZnO | DIW/Acetone | E. coli | 54.054 |
| | tube | | | 50/50, | | |
| | | | | 5% HCL 0.1M | | |
| PVC | Endotracheal | MB | ZnO | DIW/MEK | E. coli | 93.721 |
| | tube | | | 70/30, | | |
| | | | | 5% NaOH | | |
| | | | | 0.1M | | |
| PVC | Endotracheal | RB | Cu | DIW/MEK | E. coli | 93.023 |
| | tube | | | 70/30, | | |
| | | | | 5% HCL 0.1M | | |
| PVC | Endotracheal | MB | — | DIW/MEK | E. coli | 99.791 |
| | tube | | | 70/30, | | |
| | | | | 5% HCL 0.1M | | |
| PVC | Endotracheal | — | ZnO | DIW/MEK | E. coli | 94.994 |
| | tube | | | 70/30, | | |
| | | | | 5% NaOH | | |
| | | | | 0.1M | | |
| PVC | Endotracheal | — | Cu | DIW/MEK | E. coli | 99.721 |
| | tube | | | 70/30, | | |
| | | | | 5% NaOH | | |
| | | | | 0.1M | | |
| PVC | Endotracheal | MB | ZnO | DIW/MEK | S. epidermis | 99.999 |
| | tube | | | 70/30, | | |
| | | | | 5% NaOH | | |
| | | | | 0.1M | | |
| PVC | Endotracheal | MB | Cu | DIW/MEK | S. epidermis | 99.999 |
| | tube | | | 70/30, | | |
| | | | | 5% NaOH | | |
| | | | | 0.1M | | |
| PVC | Endotracheal | RB | ZnO | DIW/MEK | S. epidermis | 97.407 |
| | tube | | | 70/30, | | |
| | | | | 5% NaOH | | |
| | | | | 0.1M | | |
| PVC | Endotracheal | RB | Cu | DIW/MEK | S. epidermis | 99.999 |
| | tube | | | 70/30, | | |
| | | | | 5% NaOH | | |
| | | | | 0.1M | | |
| PVC | Endotracheal | MB | — | DIW/MEK | S. epidermis | 99.999 |
| | tube | | | 70/30, | | |
| | | | | 5% NaOH | | |
| | | | | 0.1M | | |
| PVC | Endotracheal | — | ZnO | DIW/MEK | S. epidermis | 82.222 |
| | tube | | | 70/30, | | |
| | | | | 5% NaOH | | |
| | | | | 0.1M | | |
| PVC | Endotracheal | — | Cu | DIW/MEK | S. epidermis | 99.999 |
| | tube | | | 70/30, | | |
| | | | | 5% NaOH | | |
| | | | | 0.1M | | |
| Latex | Drain | MB | Ag | DIW/Acetone | E. coli | 99.95 |
| | | | | 70/30, | | |
| | | | | 5% NaOH | | |
| | | | | 0.1M | | |
| Latex | Drain | MB | Cu | DIW/Acetone | E. coli | 99.97 |
| | | | | 70/30, | | |
| | | | | 5% NaOH | | |
| | | | | 0.1M | | |
| Latex | Drain | MB | — | DIW/Acetone | E. coli | 99.64 |
| | | | | 70/30, | | |
| | | | | 5% NaOH | | |
| | | | | 0.1M | | |
| Latex | Drain | — | ZnO | DIW/Acetone | E. coli | −25 |
| | | | | 70/30, | | |
| | | | | 5% NaOH | | |
| | | | | 0.1M | | |
| Latex | Drain | MB | Cu | DIW/MEK | E. coli | 99.999 |
| | | | | 70/30, 5% | | |
| | | | | NaOH 0.1M | | |
| Latex | Drain | RB | ZnO | DIW/Acetone | E. coli | 99.922 |
| | | | | 50/50, 5% HCL | | |
| | | | | 0.1M | | |
| POBE | Urinary | MB | ZnO | DIW/MEK | E. coli | 94.71 |
| | catheter | | | 70/30, | | |
| | | | | 5% NaOH | | |
| | | | | 0.1M | | |
| POBE | Urinary | MB | Ag | DIW/MEK | E. coli | 99.41 |
| | catheter | | | 70/30, | | |
| | | | | 5% NaOH | | |
| | | | | 0.1M | | |
| POBE | Urinary | MB | Cu | DIW/MEK | E. coli | 100 |
| | catheter | | | 70/30, | | |
| | | | | 5% NaOH | | |
| | | | | 0.1M | | |
| POBE | Urinary | MB | — | DIW/MEK | E. coli | 92.06 |
| | catheter | | | 70/30, | | |
| | | | | 5% NaOH | | |
| | | | | 0.1M | | |
| POBE | Urinary | — | ZnO | DIW/MEK | E. coli | 41.18 |
| | catheter | | | 70/30, | | |
| | | | | 5% NaOH | | |
| | | | | 0.1M | | |

### Experiment 8: Sterilisation of Antimicrobial Compounds by Ethylene Oxide and Gamma

Industry standard gamma and ethylene oxide (EtO) sterilisations were performed on a PVC-based urinary catheter to test whether sterilization affects the antimicrobial activity (FIG. 9U-V, Tables 8 and 9).

TABLE 8
Sterilisation of antimicrobial compounds using EtO.

| | | | | | | Solvent | Bacteria | % |
|---|---|---|---|---|---|---|---|---|
| Device | Type | Polymer | Dye | NP | Sterilised? | mix | Tested | Reduction |
| Urinary | EtO | PVC | MB | ZnO | Yes | DIW/Acetone | E. coli | 93.333 |
| catheter | | | | | | 70/30, 5% | | |
| | | | | | | NaOH | | |
| Urinary | EtO | PVC | MB | ZnO | Yes | DIW/Acetone | S. epidermis | 94.286 |
| catheter | | | | | | 70/30, 5% | | |
| | | | | | | NaOH | | |

EtO sterilization still leads to >90% microbial reduction across both E. coli and S. epidermis. EtO has minimal inhibitory effect to the presently disclosed technology.

TABLE 9
Sterilisation of antimicrobial compounds using gamma.

| | | | | | | | Bacteria | % |
|---|---|---|---|---|---|---|---|---|
| Device | Type | Polymer | Dye | NP | Sterilised? | Solvent mix | Tested | Reduction |
| Urinary | Gamma | PVC | MB | ZnO | Yes | DIW/Acetone | E. coli | 60.000 |
| catheter | | | | | | 70/30, 5% | | |
| | | | | | | NaOH | | |
| Urinary | Gamma | PVC | MB | ZnO | Yes | DIW/Acetone | S. epidermis | 42.857 |
| catheter | | | | | | 70/30, 5% | | |
| | | | | | | NaOH | | |

Gamma sterilisation results in <90% microbial reduction across both E. coli and S. epidermis. Gamma sterilization seems to have some inhibitory effect on the presently disclosed technology.

### Experiment 9: Biological Evaluation of Polyurethane Catheters

Cytotoxicity: The cytotoxic potential of these devices has been evaluated in accordance to BS EN ISO 10993-5. The devices were extracted in accordance with the requirements of BS EN ISO 10993-12:2021 and extracts were exposed to the NCTC Clone 929 (L-929) murine fibroblast cell line and the percentage cell viability was calculated compared to untreated cells. The devices were extracted for 23 hours and 36 minutes. The two representative Zonova devices tested had a percentage viability of 104.1% and 98.9%. Due to the percentage cell viability being >70% of the blank, this indicates both test items have no cytotoxic potential.

L-929 cells (mouse origin, connective tissue) were passaged 2 times prior to use within the test. Once confluent, cells were detached via trypsinization and adjusted to 1×105 cells/mL. 100 μL of the cell suspension was dispensed into each well of a 96-well plate to give an overall concentration of 1×104 cells/well. The seeded cells were incubated for 24 hours (5% CO2, 37±1° C., >90% humidity) so that wells formed a half-confluent monolayer. After 24 hours, the plate was examined under a phase contrast microscope to ensure that cell growth was relatively even across the wells, and any potential experimental errors were identified.

Two (2) pieces of positive control material with a total surface area of 1.54 cm2 were added to 1 mL of extraction media in a sterile container. Four (4) strips of negative control material, with a total surface area of 3.08 cm₂ was added to 1 mL of extraction media in a sterile container.

Both positive and negative controls were extracted for 23 hrs and 33 mins, as per the verified extraction time of 24±1 h, at 37±1° C.

The test item was prepared in accordance with guidance from ISO 10993-12 to identify the correct extraction ratio and incubation time for the device. The surfaces are of the devices was calculated as 83.75 cm2 and where the device thickness was >0.5 mm, an extraction ratio of 3 cm2/mL was used. For each device, 27.9 mL of extraction media was added. As the devices are intended to be used for short term contact, the devices were extracted for 23 hrs and 36 mins, within the time tolerance of 24±1 h, at 37±1° C.

Following the 24-hour incubation of the cells, the cells were examined again under a contrast microscope, and media aspirated from the wells leaving the monolayer of adhered cells on the plate. To each well, 100 μL of sample extracts, positive control, negative control or blank was added and incubated for 24 hours (5% CO2, 37±1° C., >90% humidity).

The plate was examined after the 24-hour incubation under contrast microscopy to identify and record any systematic seeding errors and the growth characteristics of the control cells. After the examination, the media in the plates was removed and 50 μL of MTT solution was added to each well. The plate was incubated for 2 hours at 37±1° C.

After the 2 hour incubation, the MTT solution was decanted and 100 μL of isopropanol was added to each well and shaken for approximately 10 minutes. The plate was then placed into the plate reader, where absorbance was read at 570 nm (reference 650 nm).

All blank control wells showed an OD570 reading of ≥0.2, which meets the acceptance criteria (Table 10). The 100% extract of the positive control had a cell viability of 1.1040% and the negative control had a cell viability of 86.2490%, both meeting the acceptance criteria. As the blank, positive and negative controls all met the acceptance criteria, this confirms the quality check of the assay, deeming the assay valid.

TABLE 10
Blank cytotoxicity results

| | Replicate 1 | Replicate 2 | Replicate 3 | Mean |
|---|---|---|---|---|
| Blank OD570 | 0.6983 | 0.5374 | 0.5908 | 0.6088 |

TABLE 11
Cytotoxicity results.

| | Measurement | | | Reference | | | | Cell |
|---|---|---|---|---|---|---|---|---|
| | OD570 | | | OD650 | | | Difference | Viability |
| | Rep 1 | Rep 2 | Rep 3 | Rep 1 | Rep 2 | Rep 3 | Mean* | (%) |
| Positive | 0.1208 | 0.1111 | 0.1254 | 0.1088 | 0.1044 | 0.1305 | 0.0045 | 1.1040 |
| 100% | | | | | | | | |
| Positive | 0.1401 | 0.0952 | 0.1205 | 0.1316 | 0.0913 | 0.1230 | 0.0033 | 0.8036 |
| 50% | | | | | | | | |
| Positive | 0.1859 | 0.1949 | 0.2008 | 0.1116 | 0.1256 | 0.1276 | 0.0723 | 17.5990 |
| 25% | | | | | | | | |
| Positive | 0.8971 | 0.8042 | 0.6190 | 0.1758 | 0.2737 | 0.2217 | 0.5497 | 133.8700 |
| 12.5% | | | | | | | | |
| Negative | 0.5491 | 0.5978 | 0.5375 | 0.2007 | 0.2234 | 0.1978 | 0.3542 | 86.2490 |
| Control | | | | | | | | |

TABLE 12
Cytotoxicity results.

| | Measurement | | | Reference | | | | Cell |
|---|---|---|---|---|---|---|---|---|
| | OD570 | | | OD650 | | | Difference | Viability |
| | Rep 1 | Rep 2 | Rep 3 | Rep 1 | Rep 2 | Rep 3 | Mean* | (%) |
| AMT100 | 0.5321 | 0.7668 | 0.6015 | 0.1642 | 0.2032 | 0.2506 | 0.4275 | 104.1000 |
| AMT010 | 0.5644 | 0.6371 | 0.4907 | 0.1519 | 0.2102 | 0.1112 | 0.4063 | 98.9450 |

The Antimicrobial Catheter AMT100 had a percentage cell viability of 104.1000%. The Antimicrobial Catheter AMT010 had a percentage cell viability of 98.9450%. Due to the percentage cell viability being >70% of the blank, this indicates both test items have no cytotoxic potential.

Toxicological: Medical devices intended for animal use only are not subject to specific regulations to prove performance and safety. However, the regulatory requirement for Medical Devices for humans provides a framework that was utilised to allow for assessment of safety. BS EN ISO 10993-1:2020 Biological Evaluation of medical devices—Part 1: Evaluation and testing within a risk management process. The guidance in this standard was used to identify toxicological risks and provide a risk assessment of the Zonova Veterinary urinary and IV catheters.

This review assessed biological risk of the Zonova Veterinary urinary and IV Catheters based on the intended use, materials of construction, manufacturing process and primary packaging materials of the final finished device. The toxicological data available did not highlight substantial risks for any of the material constituents.

Due to the product being a veterinary device, there is no regulatory requirement for the tests within ISO 10993 series to be completed. Due to the recommended tests for Irritation, Skin Sensitisation, Acute Systemic Toxicity, Material Mediated Pyrogenicity and some Haemocompatibilty tests being in vivo methods, there would not be justification ethically for these methods to be performed.

Material mediated pyrogenicity testing is waived due to none of the materials being known pyrogenic inducers and not coming from biological origin.

Haemocompatibility: The materials are also expected to exhibit haemocompatibility. Haemocompatibility of BD Insyte IV Catheter, both with the antimicrobial treatment described herein and without will be tested. PVC (empty circuit) will be used as a negative reference and flat sheet titanium as a positive reference. This will result in a total of 18 test loops (12 test loops with a IV catheter plus 6 reference test loops). Haemocompatibility of IV catheters will be evaluated by blood circulation experiments with human blood. From every group six specimens will be tested; three blood donors will be used and the catheters will be tested in duplicate per donor. Reference materials will be tested in singular per donor. IV catheters will be tested in a dynamic blood flow model (Engels G E et al. In vitro blood flow model with physiological wall shear stress for hemocompatibility testing-An example of coronary stent testing. Biointerphases. 2016; 11:031004). They will be incorporated in a closed circuit of medical grade PVC, of low activating properties (3 mm ID). The circuits are filled with heparinised blood (1.5 IU/mL) and will be circulated with a pulsatile flow for 1 h at 37° C.

Blood collected at baseline (before incubation) and after incubation will be used for analysis, see below for the assays. Additionally, a part of the whole blood will be used to prepare plasma, which will also be used for later analysis. The incubated catheters and reference materials will be gently washed with buffer, photographed (macroscopy), divided in separate pieces and stored separately. The pieces will be used to study the binding of fibrin and platelets and for SEM.

Fibrin binding assay: Adhesion of fibrin will be studied by determining the binding of labelled antibodies to the surface of the tested samples after incubation with blood. The binding of labelled antibody will expressed per unit of area and will represent the amount of bound fibrin.

Platelet binding—(activated) platelets are another major constituent of thrombi. The platelet acid phosphatase test represents surface-bound platelets and thus gives quantitative information on thrombus formation.

Macroscopic photos—will be made of the complete catheter after circulation.

SEM—Scanning electron microscopy will be used to visualize the interactions between blood components and the biomaterial of interest. The images give information about the extent of platelet, red blood cell and leukocyte adhesion, about the state of activation of the adhered platelets and about the extent of fibrin strand formation.

Activation of the clotting system—Thrombin-Antithrombin III (TAT) complexes will be determined by ELISA to measure activation of the clotting system.

Fibrinopeptide A—Fibrinopeptide A (FpA) is cleaved by thrombin from fibrinogen upon the formation of fibrin. FpA will be determined by ELISA.

Platelet release—Platelet release reaction is considered a measure of activation. Thromoxane B2 is a release product generated by activated platelets. This will be determined by means of an enzyme immunoassay in plasma.

Platelet release—Platelet release reaction is also determined by BTG release from platelet granules. This will be determined by means of an enzyme immunoassay in plasma.

Mechanically-mediated Haemolysis—An elevated level of plasma haemoglobin (HGB) after incubation of a test sample with blood indicates damage to red blood cells. Total haemoglobin concentrations in whole blood and free haemoglobin concentration in plasma samples collected before and after incubations of the test and reference samples will be determined using the direct optical method of Harboe. Extents of haemolysis will be calculated as the ratio of free-to-total haemoglobin.

### Experiment 10: Physical and Chemical Testing of Polyurethane Catheters

The physical and chemical testing of Zonova Veterinary polyurethane catheters were performed by ITA Labs (International Tin Association Limited). Company registered Number: 2994115.

Leachate Testing: Leachate tests were performed by ITA Labs after 1 week at 37° C. in water. The levels of leachate found were very low and consistent across all samples, indicating no significant chemical release from control samples to Zonova samples (Table 13).

TABLE 13
Leachate testing.

| | Nitrite | Nitrate | Sulphate | |
|---|---|---|---|---|
| ELEMENT | (ppm) | (ppm) | (ppm) | Zn (ppm) |
| Non-Sterilised PU | 1.8 | 13.4 | 27.3 | 0.004 |
| Control | | | | |
| Sterilised PU1 | 1.9 | 13.4 | 27.1 | 0.003 |
| Sterilised PU2 | 1.7 | 12.9 | 27.2 | 0.004 |

Tensile Testing: Conducted using an Instron 5550 at ITA Labs following protocol ASTM D256. The results of the testing show that the mechanical properties of the materials across control samples and Zonova samples were within experimental error (FIGS. 10A-B, 11A-B, and 12A-B). There is a slight increase in the elongation to break and the strength of the Zonova samples compared to controls, most likely associated to a change in crystallinity.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An antimicrobial composition comprising
a. one or more metallic nanoparticles selected from zinc, silver, copper, gold nanoparticles, or alloys of two or more of these metals; and
b. one or more photosensitizers selected from porphyrins, chlorins, dyes, or xanthenes;
wherein the one or more metallic nanoparticles are metal oxide nanoparticles or alloys thereof,
wherein the one or more metal oxide nanoparticles are exposed to a basic environment to form negatively charged metal ions or exposed to an acidic environment to form positively charged metal ions; and
wherein the metallic nanoparticles and the photosensitizers are mixed to form a metallic nanoparticle-photosensitizer mixture having activity in light or dark conditions without needing a light source.

2. The antimicrobial composition of claim 1, wherein the metal oxide nanoparticles are zinc oxide, silver oxide, copper oxide, gold oxide nanoparticles, or alloys thereof.

3. The antimicrobial composition of claim 1, wherein the metal oxide is a zinc oxide, silver oxide, copper oxide, gold oxide nanoparticles or alloys thereof, wherein the zinc oxide, silver oxide, copper oxide, gold oxide nanoparticles or alloys thereof are exposed to a basic environment to form negatively charged zinc oxide, silver oxide, copper oxide, or gold oxide ions that form complexes with positively charged ions, such as methylene blue and are very soluble in water, leading to much easier swell encapsulation in water-based solutions

4. The antimicrobial composition of claim 3, wherein the negatively charged zinc oxide ions are sodium zincate ions.

5. The antimicrobial composition of claim 2, wherein the metal oxide nanoparticles are exposed to an acidic environment to form positively charged zinc, silver, copper, or gold ions.

6. The antimicrobial composition of claim 5, wherein the positively charged zinc, silver, copper, or gold ions are zinc chloride, silver chloride, copper chloride, or gold chloride.

7. The antimicrobial composition of claim 1, wherein the one or more photosensitizers are selected from a group comprising porphyrins (e.g. haematoporphyrin derivatives, deuteroporphyrin), phthalocyanines (e.g. zinc, silicon and aluminum phthalocyanines), chlorins (e.g. tin chlorin e6, poly-lysine derivatives of tin chlorin e6, m-tetrahydroxyphenyl chlorin, benzoporphyrin derivatives, tin etiopurpurin), bacteriochlorins, phenothiaziniums (e.g. toluidine blue O, methylene blue, dimethylmethylene blue), phenazines (e.g. neutral red), acridines (e.g. acriflavine, proflavin, acridine orange, aminacrine), texaphyrins, cyanines (e.g. merocyanine 540), anthracyclins (e.g. adriamycin and epirubicin), pheophorbides, sapphyrins, fullerene, halogenated xanthenes (e.g. rose bengal), perylenequinonoid pigments (e.g. hypericin, hypocrellin), gilvocarcins, terthiophenes, benzophenanthridines, psoralens and riboflavin. Other possibilities are arianor steel blue, tryptan blue, crystal violet, azure blue cert, azure B chloride, azure 2, azure A chloride, azure B tetrafluoroborate, thionin, azure A eosinate, azure B eosinate, azure mix sicc. and azure II eosinate.

8. The antimicrobial composition of claim 1, wherein the photosensitizer is methylene blue or rose bengal.

9. The antimicrobial composition of claim 1, wherein the metallic nanoparticle-photosensitizer mixture comprises a zinc-methylene blue, silver-methylene blue, copper-methylene blue, or gold-methylene blue conjugate.

10. The antimicrobial composition of claim 1, wherein the metallic nanoparticle-photosensitizer mixture comprises a zinc-rose bengal, silver-rose bengal, copper-rose bengal, or gold-rose bengal conjugate.

11. The antimicrobial composition of claim 3, wherein the negatively charged zinc oxide, silver oxide, copper oxide, or gold oxide ions are catalysts that activate the photosensitizer to react with a triplet oxygen (³O₂) to form a singlet oxygen (¹O₂) or free radicals.

12. The antimicrobial composition of claim 11, wherein the singlet oxygen or free radical exhibits antimicrobial effects.

13. A method for preparing the antimicrobial metallic nanoparticle-photosensitizer mixture of claim 1 comprising contacting a solution of charge-stabilized metallic nanoparticles with a solution of photosensitizer.

14. The method of claim 13, wherein the metallic nanoparticle solution is an aqueous solution.

15. The method of claim 13, wherein the photosensitizer solution is an aqueous solution.

16. A method of encapsulating a polymer with the antimicrobial composition of claim 1 comprising:
a) dissolving the metallic nanoparticle and photosensitizer dye in a solvent mixture to form a metallic nanoparticle and photosensitizer dye mixture solution;
b) adding a pH agent to the metallic nanoparticle and photosensitizer dye mixture solution based on a v/v % of the pH agent;
c) placing the polymer into the metallic nanoparticle and photosensitizer dye mixture solution wherein there is enough metallic nanoparticle and photosensitizer dye mixture solution to cover the polymer; and
d) incubating the polymer in the metallic nanoparticle and photosensitizer dye mixture solution in dark conditions;
wherein polymer is incubated at room temperature in dark conditions for at least 12 to 18 hours.

17. The method of claim 16, wherein the solvent mixture comprises a water/acetone mixture at a ratio of 99:1, 90:10, 70:30, 60:40, or 50:50 of water to acetone.

18. The method of claim 16, wherein the polymer comprises a long chain hydrophobic polymer selected from one or more of latex, polyamide, PVC, silicones, polyethylene, polyurethane, fluoropolymer, polycarbonate, polyesters, polyofins silicone, polypropylene, fluoropolymers like polytetrafluoroethylene (PTFE), perfluoroalkoxy alkanes (PFA), fluorinatedethylenepropylene (FEP), expanded polytetrafluoroethylene (ePTFE), polyethylene terephthalate (PET), Poly(methyl methacrylate) (PMMA), hydroxyethyl methacrylate (HEMA), and combinations thereof.

19. The method of claim 16, wherein the pH agent comprises at least one base selected from sodium hydroxide (NaOH), sodium bicarbonate, potassium hydrogen carbonate, potassium hydroxide, lithium hydroxide, calcium hydroxide, strontium hydroxide, barium hydroxide, ammonia, ethylamine, pyridine, aniline, ethylamine, or ammonium hydroxide.

20. The method of claim 16, wherein the pH agent is at least one acid selected from the group comprising hydrochloric acid, phosphoric acid, nitric acid, perchloric acids, hydrobromic acid, sulfuric acid, hydroiodic acid, nitrous acid, formic acid, acetic acid, hydrocyanic acid, hydrogen or sulfide, hydrofluoric acid.
